(19) United States
(12) Patent Application Publication
(10) Pub. No.: US 2023/0093908 A1
(43) Pub. Date: Mar. 30, 2023

(54) In-situ Gel Containing Cyclosporine Micelles as Sustained Ophthalmic Drug Delivery System

(71) Applicant: IVIEW Therapeutics (Zhuhai) Co., Ltd., Zhuhai (CN)

(72) Inventors: Bo Liang, Plainsboro, NJ (US); Haizhou Peng, Wuhu (CN); Jieyu Zhu, Edison, NJ (US); Xudong Yuan, Morganville, NJ (US)

(73) Assignee: IVIEW Therapeutics (Zhuhai) Co., Ltd., Zhuhai (CN)

(21) Appl. No.: 16/975,447

(22) PCT Filed: Aug. 18, 2020
(86) PCT No.: PCT/CN2020/109682

§ 371 Date: Aug. 25, 2020

Related U.S. Application Data
(60) Provisional application No. 62/888,534, filed on Aug. 18, 2019.

(51) Int. Cl. A61K 38/13; A61K 9/107; A61K 47/36; A61K 9/00; A61K 47/26; A61K 47/10; A61K 47/02; A61K 47/14; A61K 47/18

(52) U.S. Cl. CPC A61K 38/13; A61K 9/1075; A61K 47/36; A61K 9/0048; A61K 47/26; A61K 47/10; A61K 47/02; A61K 47/14; A61K 47/18

(57) ABSTRACT

The present invention provides aqueous ophthalmic formulations containing 0.01%-5% by weight of cyclosporine which exists in the form of micelles having a particle size not greater than 20 nm, and methods of making and using such formulations.

### CROSS-REFERENCE TO RELATED APPLICATION

This application claims priority to U.S. Application No. 62/888,534, filed on Aug. 18, 2019, the contents of which are incorporated herein by reference in their entirety.

### BACKGROUND OF THE INVENTION

Dry Eye Syndrome (DES), also known as dry keratoconjunctivitis, is caused by multiple factors and complex causes, leading to abnormality in tear quality or quantity or hydrodynamic properties. It also comes with decreased tear film stability, eye discomfort and/or ocular surface tissue lesion. It is a general term for a variety of diseases which cause severe ocular surface immune inflammation and other ocular surface diseases. The most common symptoms of dry eye syndrome are burning, pain, and redness in the eyes. Other common symptoms include watery tearing or stringy mucus in the eyes. Dry eye syndrome is related to a variety of factors, the incidence rate is 7.4% ^{˜}33.7%, of which the prevalence of women over 50 years old is about twice that of men. See, e.g., JL Gayton, J. Clinical Ophthalmology (Auckland, NZ), 2009, 3: 405; D.A. Schaumberg et al., Am. J. of Ophthalmology, 2003, 136(2): 318 - 326.

Tears have three layers: an oily outer layer, a watery middle layer, and an inner mucus layer. If the glands that produce various components of tears have inflammation or don't produce enough water, oil, or mucus, it can lead to dry eye syndrome. When oil is missing from tears, the tear will quickly evaporate and is unable to maintain a steady supply of moisture. Additional common symptoms include dry eyes, eye fatigue, itchy eyes, foreign substance sensation, burning sensation, sticky secretions, sensitivities to wind, light, and other external stimuli. Sometimes the eyes are too dry to have sufficient basal tears, but are still able to stimulate the secretion of reflex tears, resulting in excessive tearing. For more severe patients, eyes will be red and swollen, with hyperemia, keratinization, corneal epithelium peeling and the subsequent adhesion of filaments. These damages can cause corneal and conjunctival lesions and affect vision. The initial symptom of dry eyes is the lack of tears to lubricate eyes. Without timely and effective treatment, it can easily develop into refractory dry eyes, leading to keratitis and corneal ulcers, and even blindness.

With the widespread use of video terminals and air-conditioning facilities in residential and commercial environments, dry eye syndrome has become a global epidemic. At present, the lack of awareness of ocular surface diseases can affect the quality of life in patients. The incidence of dry eyes may be higher and will gradually increase among younger generation as the reliance and use of technology increases.

In recent years, the prevalence of dry eye disease (the patient's percentage in the number of people at risk for dry eye disease) is approximately 5-34%. The prevalence in the U.S. is relatively low (7%). About 75 million people suffer from dry eye disease in China due to geographical and other factors. The prevalence in China is about 21-30% and the annual growth rate is about 10%. With the aging of the population, this number is expected to increase significantly in the future.

The traditional treatment for dry eye is artificial tears and Smart Plug lacrimal embolization implants. For Sjogren's syndrome, the inflammation-related dry eye, steroids or non-steroid anti-inflammatory drugs, such as corticosteroids, tetracycline, cyclosporine, etc. are used. See, e.g., J. Mohammad A-li et al., J Ophthalmic Vis Res, 2011, 6 (3): 192 - 198.

Although the pathological mechanism of dry eyes is unknown, it is generally believed that inflammation is mediated by harmful cytokines and receptors affecting the lacrimal glands and the surface of the eyeball. Based on the examination of lacrimal glands, conjunctival biopsy specimens, tear fluid, and ocular surface impression cytology in patients with dry eye syndrome, it was also revealed that the expression of inflammatory response markers such as inflammatory cell infiltration is correlated with the severity of dry eyes. Therefore, anti-inflammatory drugs and immunosuppressants can effectively treat dry eye with ocular surface inflammation.

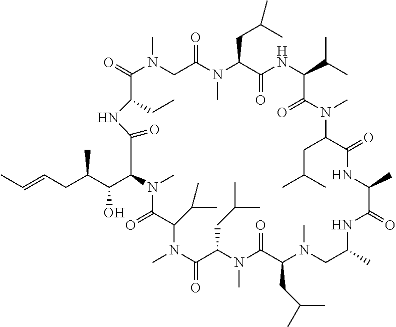 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)C(CC(C)C)C(=O)N[C@@H](C)C(=O)N[C@H](C)CN(C)[C@@H](CC(C)C)C(=O)N(C)[C@@H](CC(C)C)C(=O)N(C)C(C(C)C)C(=O)N1C]

Cyclosporine A (CsA), also called cyclosporine or cyclosporin (structure shown above), is a cyclic polypeptide compound consisting of 11 amino acids, purified from the metabolites of Trichoderma polysporumand Trichosporum. It is generally considered to be a powerful immunosuppressant. The main mechanism of cyclosporine in the treatment of dry eye is to inhibit the apoptosis of lacrimal acinar cells and conjunctival goblet cells, promote the apoptosis of lymphocytes, and inhibit ocular surface inflammation, thereby effectively treating dry eye. Systemic cyclosporine administration is affected by blood-eye barrier factors. Its ocular bioavailability is low, and it may cause complications such as renal damage, central nervous system damage, liver damage, and hypertension. Therefore, systemic cyclosporine application is greatly restricted. Topical administration methods such as eye drops can avoid these toxic and side effects.

Cyclosporine has an immunosuppressive effect and can inhibit the activation and differentiation of T lymphocytes. It mainly affects the calcineurin (CaN)/NF-AT pathway. The main mechanism is that cyclosporine selectively interacts with cyclophilin A in T cells (CyPA), and the formed CsA-CyP complex acts on CaN, inactivating CaN dephosphorylation activity, inhibiting cytoplasmic NF-AT intranuclear transfer, thereby inhibiting multiple cytokine genes like interleukin 2 (IL-2) and eventually inhibiting the differentiation and activation of T cells. After 6 months of treatment with 0.05% CsA eye drops in patients with dry eye disease, the number of conjunctival epithelial cells, CD3+, CD4+, CD8+cells, CD11a and HLA-DR cells decreased significantly (P<0.05). See, e.g., KS Kunert et al., Archives of Ophthalm., 2000, 118(11): 1489-1496. It was found in animal studies that cyclosporine inhibited the apoptosis of lacrimal acinar cells and conjunctival epithelial cells and promote lymphocyte apoptosis when treated with Sjogren-type KCA. After cyclosporine treatment, p53 protein immune activity decreased and the level of bcl-2 increased. See Gao et al., Cornea, 1998, 17(6): 654. Moore et al. established a canine keratoconjunctival xerosis model by removing the lacrimal gland. 2% cyclosporine was continuously administered for 4 weeks, and the intramucosal mucin concentration increased significantly (P<0.05). See CP Moore et al., Investigative Ophthalm. & Visual Sci., 2001, 42(3): 653-659. The symptoms of conjunctivitis were alleviated, indicating without the influence of the lacrimal gland cyclosporine has an effect on the recovery of mucin secretion function of conjunctival goblet cells, which may be an important factor for cyclosporine treatment of dry eye. The mechanism of increasing tear flow is that cyclosporine stimulates the release of neurotransmitters, Substance P, from the sensory nerve terminals, and activates muscarinic receptors through substance P, thereby increasing tear secretion. A. Yoshida et al., Exp. Eye Res., 1999, 68(5): 541-546.

US Pat. Nos. 8,629,111, 8,648,048, 8,685,930, and 9,248,191 disclose cyclosporine ophthalmic medications in emulsion forms. Restasis® 0.05% cyclosporine was developed as an emulsion formulation to increase bioavailability of cyclosporine since cyclosporine is insoluble in water. This product was marketed by Allergan and requires twice a day dosing in each eye and at least 6 weeks to show effects on dry eye improvement. The most common adverse effect following the use of RESTATIS® (cyclosporine 0.05% ophthalmic emulsion) is ocular burning as reported in 17% of patients. Other adverse reactions include conjunctival hyperemia, epiphora, eye pain, discharge, foreign body sensation, pruritic, stinging and visual disturbance (in 1-5% patients).

There was large effort to further improve bioavailability of cyclosporine to improve safety and efficacy however without much success in the past 15 years. U.S. Pat. No. 8,980,839 describes a new solution formulation of cyclosporine comprising of polyoxyl lipid or fatty acid and a polyalkoxylated alcohol in mixed nanomicelles. This led to recent commercialization of CEQUA® 0.09% Cyclosporine sterile ophthalmic solution, and it was approved in US in 2018^{[11]}. Though cyclosporine is a white powder insoluble in water, with the nanomicelle technology, CEQUA® is supplied as a clear ophthalmic solution and is able to deliver a higher concentration of cyclosporine (0.09%) into the eye compared to RESTASIS® (0.05% cyclosporine). Since then a lot of researches were dedicated to nanomicelle formulations to discover new solubilizers for cyclosporine. U.S. Pat. No. 2019/0060397 described research development on topical ophthalmic formulations containing 0.087-0.093 wt % of cyclosporine consisting of a polyoxyl lipid or a fatty acid and polyalkoxylated alcohol. Polyoxyl lipid was selected from the group consisting of HCO-40(HCO-40 is polyoxyethylene 40 hydrogenated castor oil), HCO-60, HCO-80 and HCO-100. Polyalkoxylated alcohol is also known as octoxynol 40. Bio-adhesive polymer is selected from the group consisting of Carbopol, carbophil, cellulose derivatives, gums such as xanthan gum, karaya, guar, tragacanth, agarose and other polymers such as povidone, polyethylene glycol, poloxamers, hyaluronic acid or combinations thereof. CN 104302308, CN 103735495, CN 99102848, and CN 105726479 describe cyclosporine formulations mixing with different polyoxyethylene castor oil series compounds to increase solubility of cyclosporine. However, these patents do not have significant difference regarding solubilizers. CN 103054796 described Soluplus as a solubilizer, and its formed particle size was around 60 nm. U.S. Pat. No. 2009/0092665 discloses drug delivery systems to form nanomicelle using Vitamin-E TPGS. Polyoxyethylene hydrogenated castor oil series surfactants are used in these patents, however no surfactants have been found that could produce smaller size of cyclosporine micelles than 20nm.

Drugs penetrate through the corneal epithelium mainly through transcellular and paracellular pathways, based on their lipophilicity and hydrophilicity (see, e.g., E. Toropainen et al., European J. of Pharmaceutical Sciences, 2003, 20(1): 99-106). Hydrophilic compounds are permeated via paracellular pathways, which is influenced by paracellular porosity and pore sizes, while the permeation of intermediate and hydrophobic compounds are through epithelial transcellular pathways and stromal pathways, respectively (see A. Edwards et al., Pharm. Res., 2001, 18(11): 1497-1508). Cyclosporine A (CsA) is a neutral, lipophilic, cyclic endecapeptide. Without any encapsulation, CsA is absorbed through transcellular pathways (see K. Kawazu et al., Investigative Ophthalm. & Visual Sci., 1999, 40(8): 1738-1744). But once it is encapsulated in micelles, the hydrophilic surface of micelles makes the paracellular route the dominant pathway.

A large number of relevant research materials on the use of nanotechnology to increase the corneal permeability of poorly soluble drugs (see F. Bongiovi et al., Macromol Biosci. 2017;17(12):10.1002). These documents all show that the preparation of poorly soluble drugs in nanoparticles can significantly increase the permeation efficiency of the drug in the cornea and increase bioavailability, including the preparation of micellar solutions, microemulsion solutions, nano suspensions and emulsions, etc. The smaller the nano particle size, the higher the corneal permeability and the higher the bioavailability. Factors such as the preparation of micellar solutions, micro-emulsions, nano-suspensions and emulsions that contain small nano particle size will have a higher corneal permeability and higher bioavailability.

Micelles are amphiphilic colloidal structures, with particle diameters from 5 to 100 nm range (See M. Milovanovic et al., Nanoparticles in Antiviral Therapy: Antimicrobial Nanoarchitectonics, Chapter 14, 2017, p.383-410.) However, nanomicelle formulations with particle size less than 20nm are never able to be prepared and reported. Therefore, it's our goal to further reduce micelle sizes by discovering novel powerful solubilizers or combinations and improve the permeation of cyclosporine in the eyes.

RESTASIS® developed by Allergan is an ophthalmic emulsion with an average particle size around 160 nm. It has poor mucosal adhesion and short corneal retention time. Therefore, the bioavailability is low and its therapeutic effect is not ideal. Moreover, it is irritating to eyes and causes undesirable symptoms such as foreign substance sensation which is not easily tolerated by patients. CEQUA® developed by Sun Pharmaceutical is a micellar eye drop with an average particle size around 25 nm, but the bio-adhesion of micellar eye drops is similar to that of traditional eye drops. It cannot adhere to the eye for a long period of time and cannot overcome the drug loss caused by nasolacrimal drainage. Although the micellar solution increases the permeability of the cyclosporine to the cornea, the rapid loss in the eye prevents the increase of its bioavailability.

### BRIEF SUMMARY OF THE INVENTION

To solve these problems, we have developed, with newly discovered solubilizers or surfactants, new nano-carriers that can carry cyclosporine to form extremely small nanomicelles. Because of their small size, these nanocarriers can carry higher concentrations of cyclosporine into the cornea and conjunctiva cells, resulting in an increase in drug efficacy. It was surprising that some newly discovered solublizers or surfactants be combined with in-situ gel technology using polysaccharide polymers to form an in-situ gel when instilling the eye drop into the eyes, thus increasing drug retention time on the eye surface and further increasing the bioavailability of the drug in the eyes. Additionally, in-situ gel sustained-release technology further reduces adverse reactions such as local irritation, pain and foreign body sensation in the eyes.

The in-situ gel delivery system can prolong the retention time of the drug on the cornea surface, which helps to improve the bioavailability of the drug in the eye. Ideally, the in-situ gel system is a low-viscosity, free-flowing liquid during storage, which allows the eye drops to be used repeatedly and easily on the eye. After administration on the conjunctival sac, it forms an in-situ gel which adheres to the surface of the eye. The viscosity of the in-situ gel should be sufficient to withstand the shear forces in the eye and prolong the retention time of the drug in the front of the eye. Slowly-released drugs can help improve bioavailability, reduce systemic absorption, reduce the frequency of medications, and thereby improving patient compliance. However, using an in-situ gel system can increase the retention time of the drug in the eye and prolong the absorption of the drug. For water insoluble drug substances, it's challenging to achieve overall sufficient bioavailability of those molecules with poor aqueous solubility. As such, it was our goal to develop the in-situ gel forming formulation containing cyclosporine as the active ingredient with novel solubilizers or surfactants to achieve significant permeation increase for enhanced efficacy and reduced side effects in humans.

Micellar surfactants are dissolved and adsorbed to the drug molecules at low concentrations in water. When the concentration of the surfactant is increased to the point where the molecule surface is saturated and cannot be adsorbed again, the surfactant molecules begin to accumulate in the solution. Because the hydrophobic part of the surface-active molecule has less affinity with water and the attraction between the hydrophobic parts is larger, the hydrophobic parts of many surfactant molecules attract and associate with each other thereby forming a multi-molecular or ionic composite, which is known as micelle. This nano-micelle formulation allows cyclosporine molecules to overcome solubility challenges, allowing the penetration through the aqueous layer of the eye and the prevention of rapid release of active lipophilic molecules before penetration. The micelles have a particle size much smaller than that of ordinary emulsions. They can penetrate into the cornea more effectively, thereby enhancing drug efficacy and greatly improving its bioavailability.

In the current invention, we developed in-situ gel forming cyclosporine formulations with nanomicelle delivery systems, so that the new composition can improve the drug's membrane transportation through the nano-carrier, increase drug permeability to the biofilm while improving the drug's stability, solubility, and provide targeted delivery. In addition, the current invention can also increase the adhesiveness of the eye drops through the in-situ gel drug delivery system and further improve the drug retention time on the surface of cornea. The successful combination of in-situ gel and nanomicelle delivery system overcomes the shortcomings of using a single formulation delivery technology. Comparing to the current nanomicelle or emulsion drug delivery system for cyclosporine, the nanomicelle in-situ gel drug delivery system offers significant advantages.

Accordingly, one aspect of the present invention provides micelles each comprising water, a cyclosporine, and a solubilizer, wherein the micelle has a particle size no greater than 20 nm. Examples of a suitable solubilizer include Polyoxyl 20 Cetostearyl Ether, Polyoxyl 15 Hydroxystearate, Soluplus, Polyoxyethylene hydrogenated castor oil, Polyoxyethylene castor oil, Vitamin E Polyethylene Glycol Succinate, and any combination thereof; and a suitable example of the cyclosporine is cyclosporin A. The cyclosporin can be contained in the formulation at a concentration suitable for the intended use, e.g., at a concentration of 0.01% to 5% by weight.

In another aspect, the present invention provides an aqueous ophthalmic formulation which includes a cyclosporine, a solubilizer, an osmotic pressure regulator, a pH regulator, a viscosity adjuster, and water, wherein micelles with particle size no greater than 20 nm are formed with cyclosporine and the solubilizer and contained in the formulation.

In some embodiments, the aqueous ophthalmic formulation further includes a gel-forming polysaccharide polymer, and a gel is formed in situ at the physiological temperature with instant viscosity increase upon instillation of the formulation into the eye. The polysaccharide can be contained in the formulation at a concentration of 0.1% to 0.6% by weight. Examples of a polysaccharide suitable for the formulation of this invention include deacetylated gellan gum (DGG), xanthan, sodium alginate, carrageenan, or any mixture thereof. In some further embodiments, the polysaccharide includes deacetylated gellan gum.

In still some other embodiments, a solubilizer suitable for the present invention, as example, is Polyoxyl 20 Cetostearyl Ether, Polyoxyl 15 Hydroxystearate, Soluplus, Polyoxyethylene hydrogenated castor oil, Polyoxyethylene castor oil, Vitamin E Polyethylene Glycol Succinate, or any combination thereof. The solubilizer can be contained in the formulation at a concentration of 0.01% to 10% by weight.

In some embodiments, the osmotic pressure regulator contained in the formulation of the present invention includes sodium chloride, mannitol, glucose, sorbitol, glycerin, polyethylene glycol, propylene glycol, or any combination thereof. Such an osmotic pressure regulator can be contained in the formulation at a concentration of 0.01% to 10% by weight.

The formulations of the present invention may further include a preservative which may include, e.g., butylparaben, benzalkonium chloride, benzalkonium bromide, chlorhexidine, sorbate, chlorobutanol, or any combination thereof. As an example, the preservative in the formulation can be at a concentration of 0.01% to 5% by weight.

In some embodiments, the pH adjuster contained in the formulations of the present invention comprises boric acid, sodium borate, phosphate buffer, tromethamine, tromethamine hydrochloric acid buffer, sodium hydroxide, hydrochloric acid, citric acid, sodium citrate, or any combination thereof. The pH adjuster contained in the formulation can have a concentration of 0.01% to 5% by weight.

In some embodiments, the viscosity adjuster in the formulation has a concentration of 0.01% to 5% by weight. Examples of a suitable viscosity adjuster include carboxyl methyl cellulose, sodium cellulose, hydroxypropyl cellulose, hydroxyethyl cellulose, and any combination thereof.

In some embodiments, the average particle size of the micelles contained in the formulations of the present invention ranges from 10 nm to 20 nm.

Still another aspect of the invention provides a method of treating or alleviating symptoms of dry eye disease or condition in a subject in need thereof, wherein the method includes administering to the eye of the subject a therapeutically effective amount of an aqueous ophthalmic formulation or micelles as described above.

### BRIEF DESCRIPTIONS OF THE DRAWINGS

FIG. 1 shows the particle size and distribution of Sample 1 prepared in Example 1.

FIG. 2 shows the particle size and distribution of Sample 2 prepared in Example 1.

FIG. 3 shows the particle size and distribution of Sample 3 prepared in Example 1.

FIG. 4 shows the particle size and distribution of Sample 4 prepared in Example 1.

FIG. 5 shows the particle size and distribution of Sample 5 prepared in Example 1.

FIG. 6 shows the particle size and distribution of Sample 6 prepared in Example 1.

FIG. 7 shows the particle size and distribution of Sample 7 prepared in Example 1.

FIG. 8 shows the particle size and distribution of Sample 8 prepared in Example 1.

FIG. 9 shows the bar chart of viscosity changes of formulation Sample 1 to Sample 6 with gelling matrix DGG prepared in Example 2.

FIG. 10 shows the bar chart of viscosity changes of formulation Sample 7 to Sample 10 with gelling matrix xanthan gum prepared in Example 2.

FIG. 11 shows the bar chart of viscosity changes of formulation Sample 11 to Sample 14 with gelling matrix carrageenan prepared in Example 2.

FIG. 12 shows the bar chart of viscosity changes of formulation Sample 15 to Sample 18 with gelling matrix sodium alginate prepared in Example 2.

FIG. 13 shows the particle size and distribution of the sample prepared in Example 3.

FIG. 14 shows the particle size and distribution of RESTASIS.

FIG. 15 shows the particle size and distribution of CEQUA.

FIG. 16 shows in vitro release curve of the sample prepared in Example 3, RESTASIS®, CEQUA®.

FIG. 17 shows the particle size and distribution of the sample prepared in Example 4.

FIG. 18 shows the in vitro release curve of the sample prepared in Example 4, RESTASIS®, CEQUA®.

FIG. 19 shows the particle size and distribution of the sample prepared in Example 5.

FIG. 20 shows the in vitro release curve of the sample prepared in Example 5, RESTASIS®, CEQUA®.

FIG. 21 shows the particle size and distribution of the sample prepared in Example 6.

FIG. 22 shows the in vitro release curve of the sample prepared in Example 6, RESTASIS®, CEQUA®.

FIG. 23 shows the particle size and distribution of the sample prepared in Example 7.

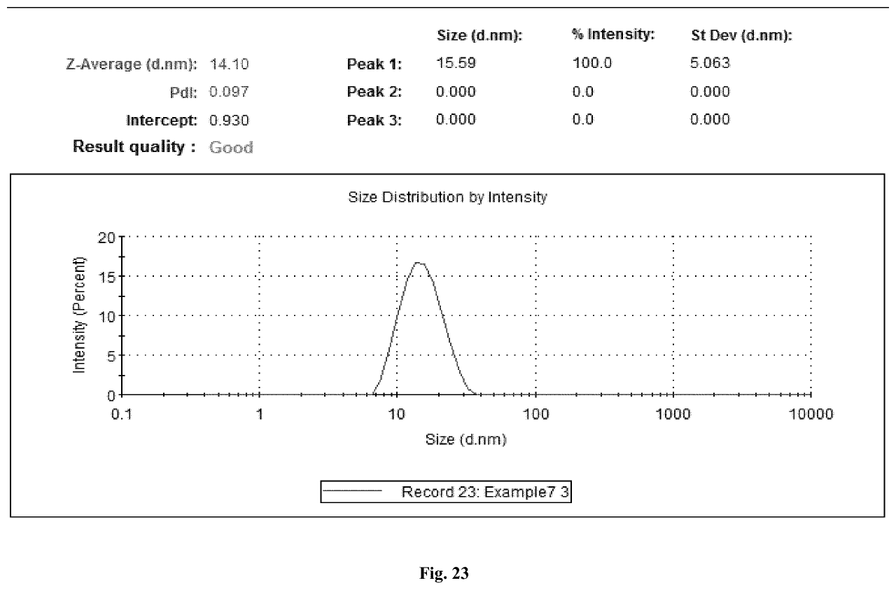

FIG. 24 shows the in vitro release curve of the sample prepared in Example 7, RESTASIS®, CEQUA®.

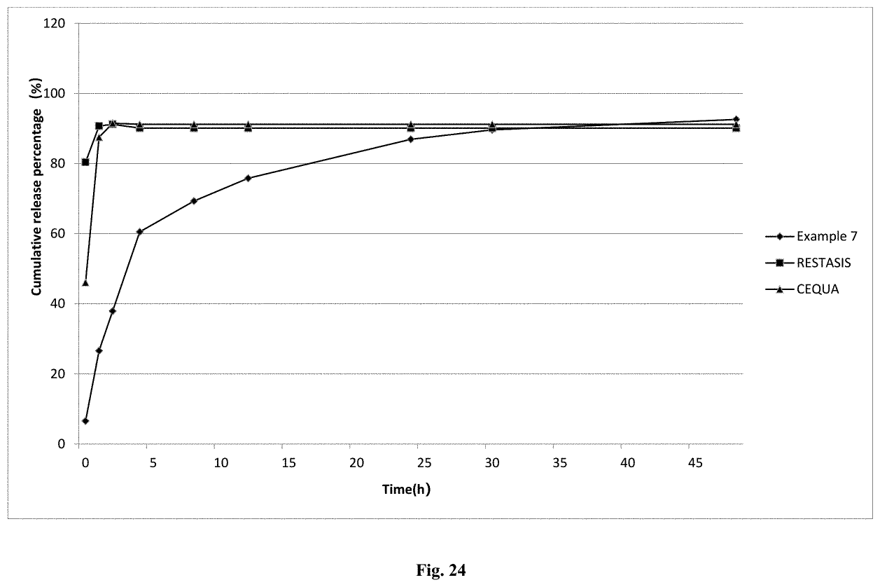

FIG. 25 shows the in vitro dialysis release test of the sample prepared in Example 8 (Samples 1-3), RESTASIS®, CEQUA®.

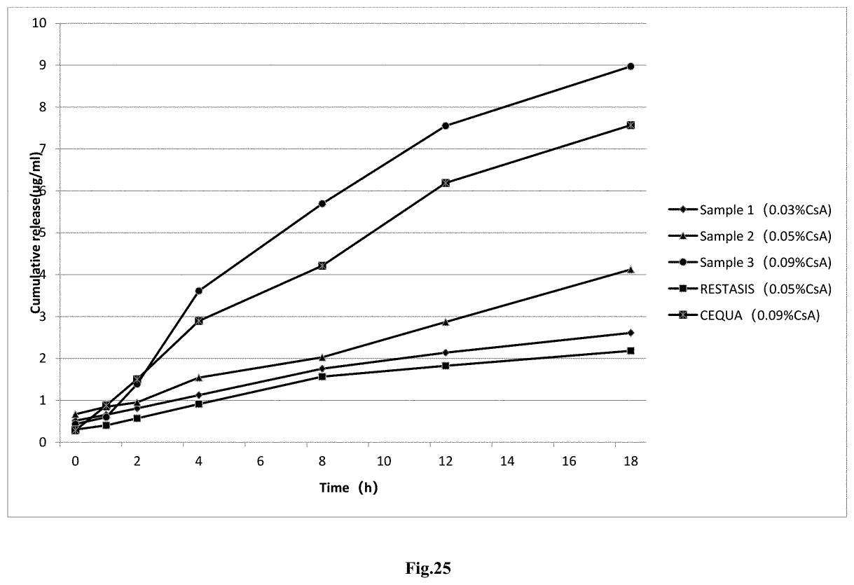

FIG. 26 shows the in vitro dialysis release test of the sample prepared in Example 8 (Samples 4-6), RESTASIS®, CEQUA®.

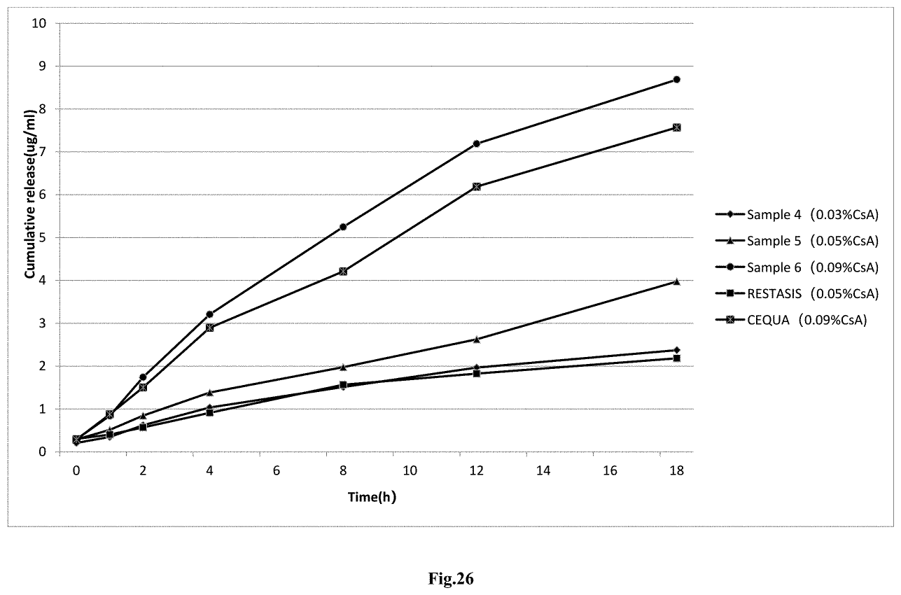

### DETAILED DESCRIPTION OF THE INVENTION

The solubilizers that were used to prepare cyclosporine into micellar solutions as described in literature have been investigated, but were found that the particle sizes formed in those formulations were all above 20 nm. U.S. Pat. No. 2019/0060397A1 describes the use of HCO (i.e., polyoxyethylene hydrogenated castor oil) combined with octoxynol 40 to form a micellar solution, we have confirmed that the particle size of CEQUA® is 22 nm. U.S. Pat. No. 2009/0092665 describes micellar solutions prepared using vitamin E TPGS as a solubilizer and its particle size was larger than 20 nm. CN 103735495B describes the use of polyoxyethylene castor oil as a solubilizer to prepare a micellar solution. Similarly, the micellar solution forms particle size larger than 20 nm. In all the examples mentioned above as cyclosporine solubilizers, the particle sizes formed were all above 20 nm (See Table 1).

TABLE 1
The particle size of micelles prepared
by solubilizers reported in prior arts

| | | Percentage | Particle size |
|---|---|---|---|
| | Solubilizer | (w/w %) | (nm) |
| 0.05% CsA | Polyoxyethylene | 1.0%/0.05% | 22 nm |
| | hydrogenated 40 | | |
| | castor oil/Octoxynol 40 | | |
| | Polyoxyethylene | 10% | 60 nm |
| | castor oil 60 | | |
| | Vitamin E TPGS | 3% | 30 nm |

In order to further increase the bioavailability of cyclosporine in the eye, we have conducted a large number of experiments. We have surprisingly found several solubilizers or combinations of some solubilizers unexpectedly resulted in formation of cyclosporine-containing micelles with particle size less than 20 nm.

In one aspect, one type of suitable solubilizers is Cetomacrogol 1000 series which has the formula of CH₃[CH₂]ₘ[OCH₂CH₃]ₙOH, with n being 20^{˜}24 and m being 15^{˜}17. Based on the quantity of ethylene oxide (n), it has 2 CAS numbers: CAS 9004-95-9 (macrogol cetyl ethers); CAS 68439-49-6 (macrogol cetostearyl ethers). One representative ingredient of Cetomacrogol 1000 series, Polyoxyl 20 Cetostearyl Ether, belongs to polyoxyethylene (20) cetyl octadecyl ether (n=20) in the polycetol 1000 series. Polyoxyl 20 cetostearyl ether is used as an emulsifier in creams (Synalar®). It had never been reported as a solubilizer for ophthalmic preparations, and there is no research on it as a solubilizer for cyclosporine to form a micellar solution. We have surprisingly discovered that polyoxyl 20 cetostearyl ether (solubilizer A) can form a micellar solution with cyclosporine above its critical micelle concentration for ophthalmic application. Additionally, we have surprisingly found out that the sample's particle average size was extremely small at around 10 nm and maintains uniformity and stability. The particle sizes of these samples were much smaller than those of RESTASIS® and CEQUA®. We expect to have a higher corneal permeability compared to RESTASIS® and CEQUA®, therefore increasing the bioavailability.

In another aspect, Polyoxyl 15 Hydroxysterate is used as an emulsifier in microemulsion ophthalmic preparations. For example, the commercial product Xelpros® contains 0.25% of Polyoxyl 15 hydroxystearate. CN 201510785005.4 discloses use of Polyoxyl 15 hydroxystearate as an emulsifier at the concentration of 1.2%^{˜}3.5%. In another prior art example, the particle size of microemulsions prepared with the emulsifier polyoxyl 15 hydroxysterate is 50±30 nm (See L. Gan et al., Int J Pharm., 2009; 365 (1-2): 143-149.). The cyclosporine microemulsion solution prepared by using polyoxyl 15 hydroxystearate as an emulsifier had a particle size greater than 20 nm. Polyoxyl 15 hydroxystearate was never reported to be used as a solubilizer for ophthalmic preparations to prepare micellar solution. The maximum safe dosage of polyoxyl 15 hydroxystearate as an emulsifier for ophthalmology is 0.25%. We have confirmed in our own experiments that 0.25% polyoxyl 15 hydroxystearate could only serve as an emulsifier and could not result in formation of a micellar solution with 0.05% CsA. But we were surprised to discover that polyoxyl 15 hydroxystearate at 1.0% resulted in formation of a micellar solution with cyclosporine above its critical micelle concentration. It was discovered that the sample's particle size was very small, ranging from 10 nm to 15 nm, therefore maintaining good uniformity and stability.

In another aspect, Soluplus (polyethylene caprolactam-polyvinyl acetate-polyethylene glycol graft copolymer) is a new type of solubilizer, which is mostly used in oral solid preparations. Soluplus has not been used in any commercial eye drops. We surprisingly found out that Soluplus with a concentration of 0.9% and above resulted in forming a micellar solution with 0.05% CsA, and the micelles formed at different concentrations of Soluplus had a particle size of about 65 nm. On the basis of this micellar solution, we also surprisingly discovered that this micellar solution could be combined with the in-situ gel to form micellar in-situ gel eye drops which increased the retention time of micellar particles on the ocular surface and improved bioavailability, and the solution was stable.

Based on our experimental results, a suitable solubilizing system was found to be any combinations of polyoxyl 20 cetostearyl ether, polyoxyl 15 hydroxystearate, polyoxyethylene hydrogenated castor oil, polyoxyethylene castor oil, and vitamin E polyethylene glycol succinate. It was found that these combinations also had a good solubilizing capacity for cyclosporine which could form micelles with particle sizes smaller than 20 nm.

The above solubilizers or mixtures thereof were used with 0.09% cyclosporine to investigate their solubilizing ability. These solubilizers or their mixtures were also found to have a good solubilizing effect for cyclosporine. The particle size of the resultant micelles was much smaller than the particle size of micelles prepared with RESTASIS® or CEQUA®.

The in-situ gel forming cyclosporine nanoparticle carrier are formulated with one or more ion-sensitive in-situ gel forming materials such as polysaccharides to increase the residence time of the dosage form in the eyes. An in-situ gel topical drug delivery platform was developed by employing an ion-sensitive polysaccharide (e.g., gellan gum) as the gel-forming matrix. Different concentrations of gellan gum were used to determine the viscosity changes at 25° C. (without artificial tears) and 34° C. (with artificial tears), to produce in vitro release profile. Only such optimized gel matrix can potentially form an in-situ gel.

Deacetylated gellan gum (“DGG”, an exocellular polysaccharide of microbial origin, commercially available as Gelrite®) is an interesting in-situ gelling polymer that seems to perform very well in humans. DGG is an anionic linear polysaccharide comprised of a plurality of four-sugar units. Upon instillation of DGG solutions containing drugs into eyes, gel is formed in-situ after interaction of DGG with the electrolytes (Na⁺, K⁺, Ca²⁺, etc.) in the eye fluid. Since human eye fluid contains large amounts of ions (e.g., sodium, potassium, and calcium ions), ion-sensitive gel preparations are expected to achieve a solution-gel phase transition.

The current invention involves the incorporation of cyclosporine nano-micelles in the in-situ gel matrix and the formulations are further optimized with the following iterative approaches.

The current invention is further elucidated with specific examples. It is understood that these examples are included herein to illustrate, and not intended to limit the scope of, the invention. The experimental methods with no specific conditions in the following examples are usually prepared under conventional conditions as reported in the literature or according to the conditions suggested by the excipient's manufacturer. Unless specifically stated, all percentages, ratios, proportions or fractions in this invention are calculated on the weight-by-weight basis. Unless specifically defined in this invention, all professional and scientific terms used herein have the same meaning as well-trained personnel may be familiar with. In addition, any methods and materials similar or equivalent to those recorded in this invention can be applied to this invention. The preferred embodiments and materials described herein are used only for exemplary purposes.

### Example 1: Determination of Concentration of Solubilizer

Samples of the micelle solution s containing 0.05% cyclosporin A are listed in Table 2 below:

TABLE 2
Sample Formulations of Cyclosporine A Nanomicelle Solutions

| Sample | Active | Concentration of | | Concentration of |
|---|---|---|---|---|
| No. | ingredients | active ingredients | Solubilizer | solubilizer |
| 1 | Cyclosporine | 0.05% | Polyoxyl 20 Cetostearyl Ether | 0.6% |
| 2 | A | | (Solubilizer A) | 5.0% |
| 3 | Cyclosporine | 0.05% | Polyoxyl 15 hydroxystearate | 0.6% |
| 4 | A | | (Solubilizer B) | 5.0% |
| 5 | Cyclosporine | 0.05% | Soluplus | 0.9% |
| 6 | A | | (Solubilizer C) | 5.0% |
| 7 | Cyclosporine | 0.05% | Polyoxyl 15 hydroxystearate/ | Polyoxyl 15 |
| | A | | Hydrogenated 40 castor oil | hydroxystearate: |
| | | | (Solubilizer D) | 5.0% |
| | | | | Hydrogenated 40 |
| | | | | castor oil: 0.1% |
| 8 | | | | Polyoxyl 15 |
| | | | | hydroxystearate: |
| | | | | 0.1% |
| | | | | hydrogenated 40 |
| | | | | castor oil: 5.0% |

### Particle Size and Distribution Detection

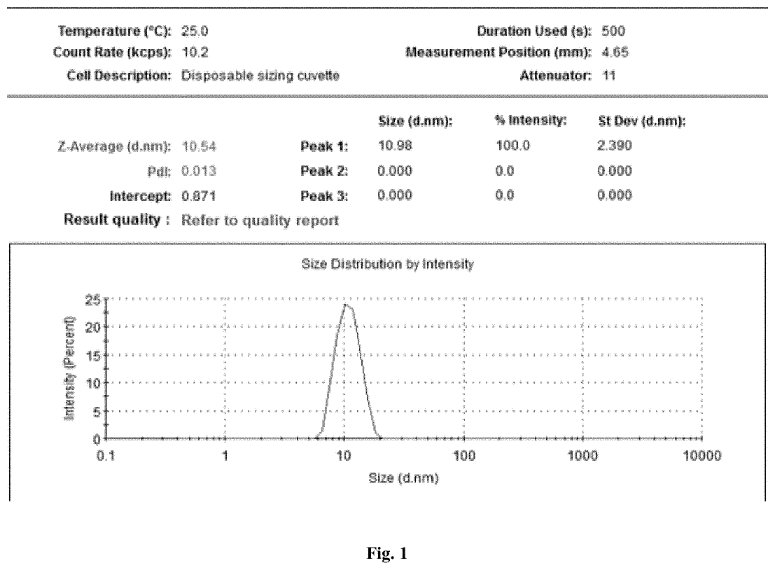

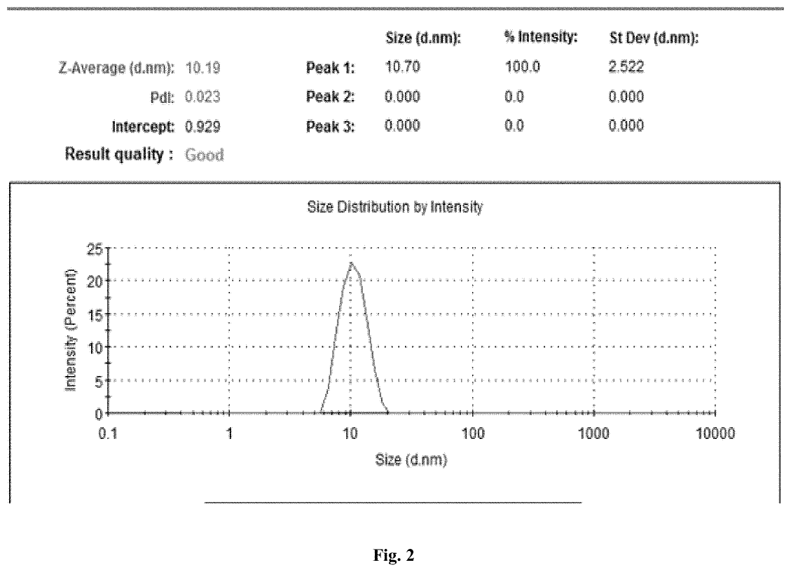

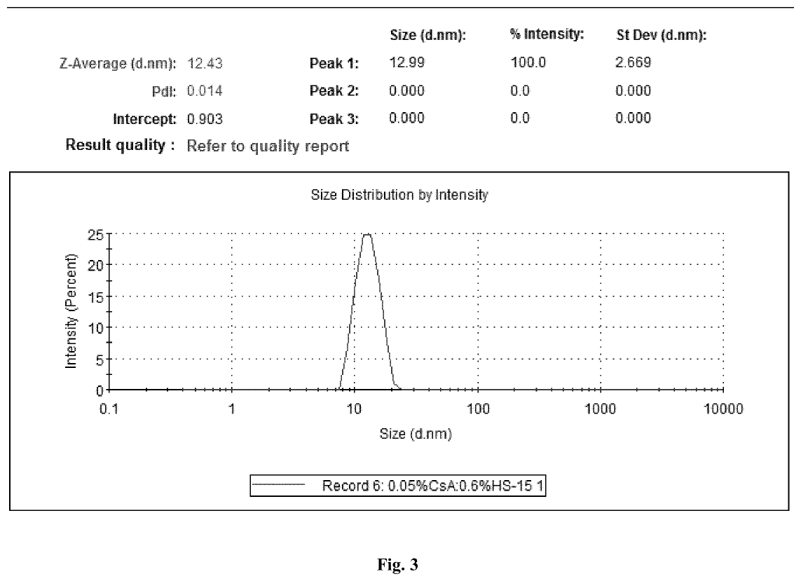

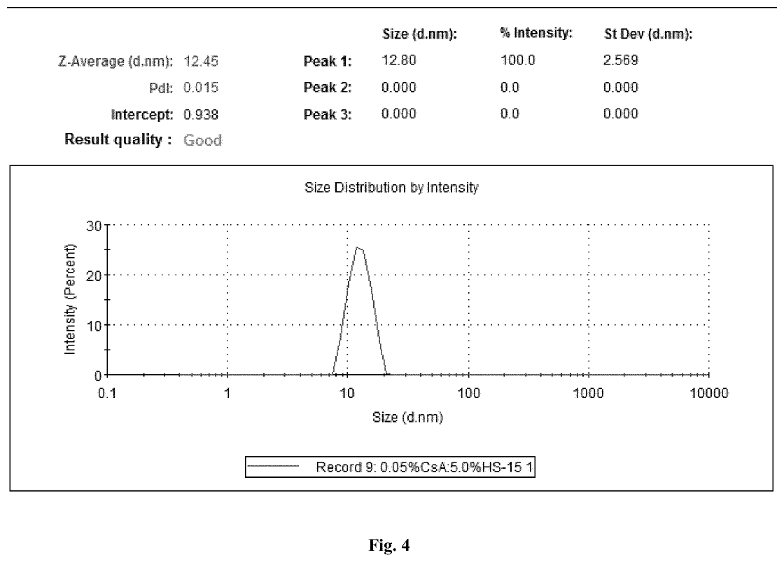

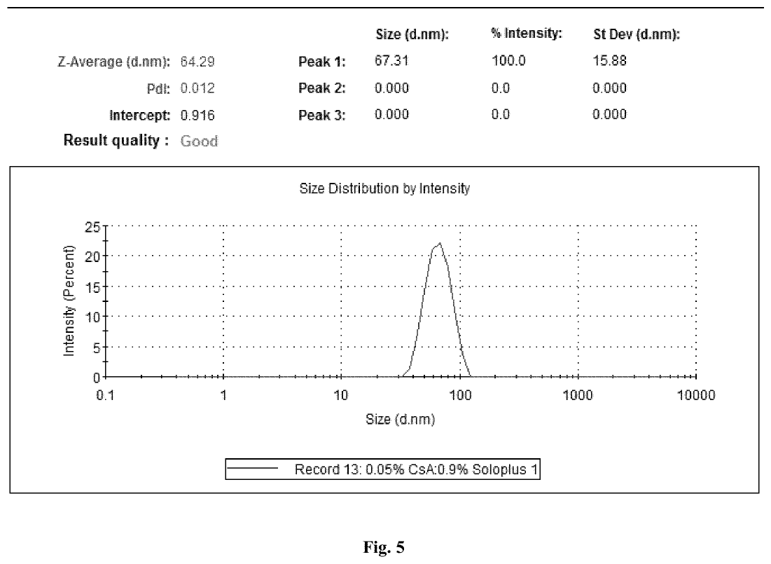

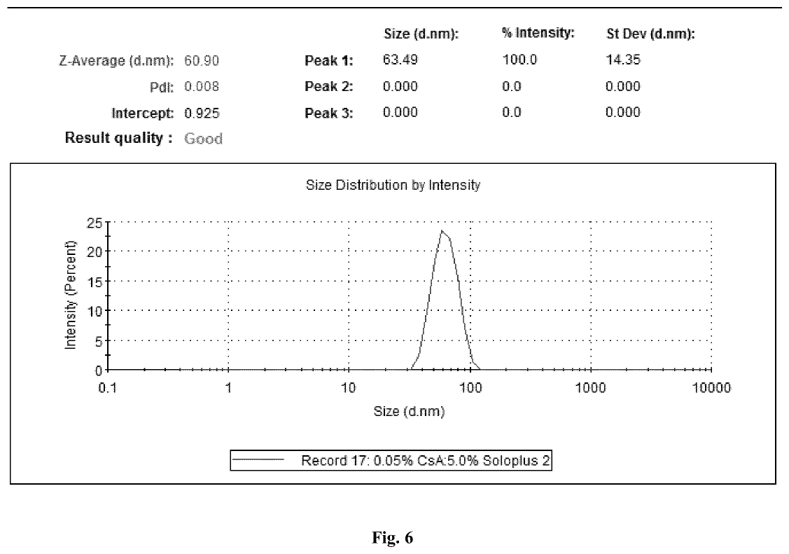

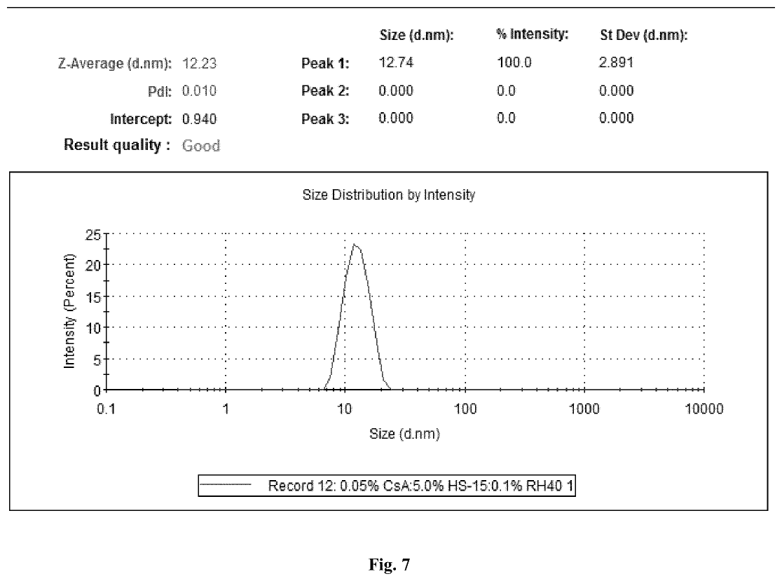

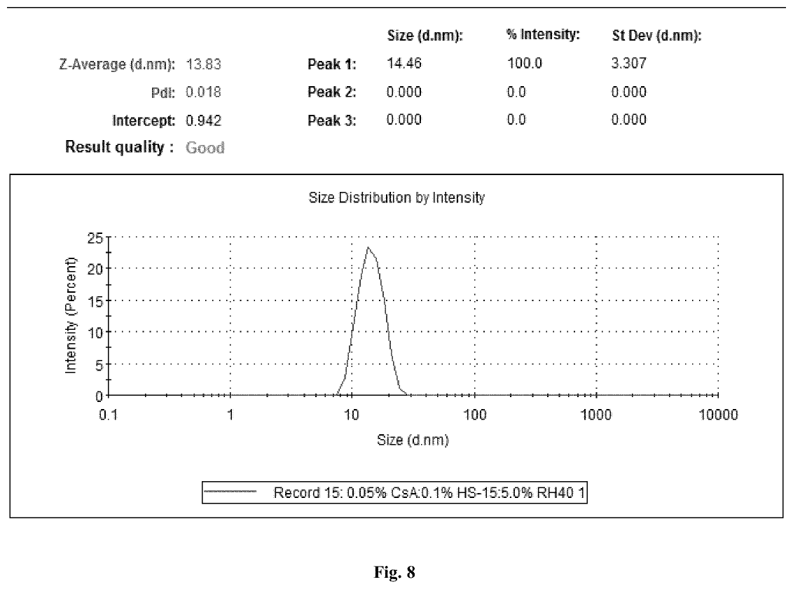

Samples 1 to 8 prepared with the above formulations were tested with a particle size analyzer for their micelle particle size and distribution or polydispersity index (PDI) (Table 3). The results are shown in FIGS. 1-8 and confirm the particle sizes of micelles in Samples 1-8 prepared and tested as described are smaller than those in RESTASIS® or CEQUA®.

TABLE 3
Comparison of particle size of nanomicelles in Samples
and RESTASIS ® and CEQUA ®

| Samples | Particle size(nm) | PDI |
|---|---|---|
| Samples 1 | 10.54 | 0.013 |
| Samples 2 | 10.19 | 0.023 |
| Samples 3 | 12.43 | 0.014 |
| Samples 4 | 12.45 | 0.015 |
| Samples 5 | 64.29 | 0.012 |
| Samples 6 | 60.90 | 0.008 |
| Samples 7 | 12.23 | 0.010 |
| Samples 8 | 13.83 | 0.018 |
| RESTASIS ® | 159.4 | 0.433 |
| CEQUA ® | 22.04 | 0.367 |

### Example 2: Determination of Concentrations of Gelling Agent

Different in-situ gelling solution samples containing 0.05% cyclosporin A are listed below in Tables 4-7:

TABLE 4
Concentrations of Gelling Agent DGG

| Sample | deacetylated gellan gum | NaCl | Cyclosporin A |
|---|---|---|---|
| 1 | 0.2% | 0.2% | 0.05% |
| 2 | | 0.3% | 0.05% |
| 3 | 0.3% | 0.2% | 0.05% |
| 4 | | 0.3% | 0.05% |
| 5 | 0.4% | 0.2% | 0.05% |
| 6 | | 0.3% | 0.05% |

TABLE 5
Concentrations of gelling agent Xanthan gum

| Sample | Xanthan gum | NaCl | Cyclosporine A |
|---|---|---|---|
| 7 | 0.1% | 0.2% | 0.05% |
| 8 | | 0.3% | 0.05% |
| 9 | 0.3% | 0.2% | 0.05% |
| 10 | | 0.3% | 0.05% |

TABLE 6
Concentration of gelling agent Carrageenan

| Sample | Carrageenan | NaCl | Cyclosporine A |
|---|---|---|---|
| 11 | 0.1% | 0.2% | 0.05% |
| 12 | | 0.3% | 0.05% |
| 13 | 0.3% | 0.2% | 0.05% |
| 14 | | 0.3% | 0.05% |

TABLE 7
Concentration of Gelling Agent Sodium Alginate

| Sample | Sodium alginate | NaCl | Cyclosporine A |
|---|---|---|---|
| 15 | 0.1% | 0.2% | 0.05% |
| 16 | | 0.3% | 0.05% |
| 17 | 0.3% | 0.2% | 0.05% |
| 18 | | 0.3% | 0.05% |

### Method for Preparation of Gel Solutions

Accurately weigh a certain amount of sodium chloride, slowly and evenly add the 85 g of ultrapure water. Stir the solution until sodium chloride was completely dissolved, then slowly and evenly add the gelling agent described above under continuous stirring. Put this solution in a 90° C. water bath and stir for 1 hour. Then cool the mixture to room temperature. Weigh 0.05 g of cyclosporin A and slowly add it to the cooled solution that is being stirred. Add water to the final quantity of 100 g.

### Artificial Tear Preparation Method

Measure NaHCO₃: 2.18 g; NaCl: 6.78 g; CaCl₂·2H₂O: 0.084 g; KCl:1.38 g. respectively and dissolve in 1,000 mL deionized water.

### Viscosity Testing Method

20 mL of sample solution was loaded to the sample cylinder and was allowed to rest for 5 minutes. Then rotate the rotor to measure the initial viscosity value at 25° C. Under 34° C. (add artificial tears-40:7): 20 mL of sample solution was loaded to the sample cylinder and held it for 5 minutes. Then rotate the rotor to measure the initial viscosity value.

Viscosities of Samples 1 to 18 were measured for values before and after adding artificial tears using a viscometer respectively. Results are shown in Tables 8-11.

TABLE 8
Viscosity of Samples 1-6

| | 25° C. | 34° C. Viscosity |
|---|---|---|
| | Viscosity | (artificial tears) |
| Sample | (mpa · s) | (mpa · s) |
| Sample 1 | 40.57 | 58.10 |
| Sample 2 | 99.70 | 369.46 |
| Sample 3 | 71.71 | 295.47 |
| Sample 4 | 238.12 | 442.28 |
| Sample 5 | 150.58 | 553.55 |
| Sample 6 | 130.91 | 583.73 |

TABLE 9
Viscosity of Samples 7-10

| | 25° C. | 34° C. Viscosity |
|---|---|---|
| | Viscosity | (artificial tears) |
| Sample | (mpa · s) | (mpa · s) |
| Sample 7 | 19.24 | 20.76 |
| Sample 8 | 19.45 | 23.21 |
| Sample 9 | 222.51 | 256.80 |
| Sample 10 | 221.68 | 255.64 |

TABLE 10
Viscosity of Samples 11-14

| | 25° C. | 34° C. Viscosity |
|---|---|---|
| | Viscosity | (Artificial tears) |
| Sample | (mpa · s) | (mpa · s) |
| Sample 11 | 0.00 | 16.16 |
| Sample 12 | 2.89 | 16.58 |
| Sample 13 | 3.20 | 19.41 |
| Sample 14 | 3.17 | 23.73 |

TABLE 11
Viscosity of Samples 15-18

| | 25° C. | 34° C. Viscosity |
|---|---|---|
| | Viscosity | (artificial tears) |
| Sample | (mpa · s) | (mpa · s) |
| Sample 15 | 4.18 | 17.84 |
| Sample 16 | 4.94 | 16.91 |
| Sample 17 | 6.87 | 26.98 |
| Sample 18 | 9.81 | 18.33 |

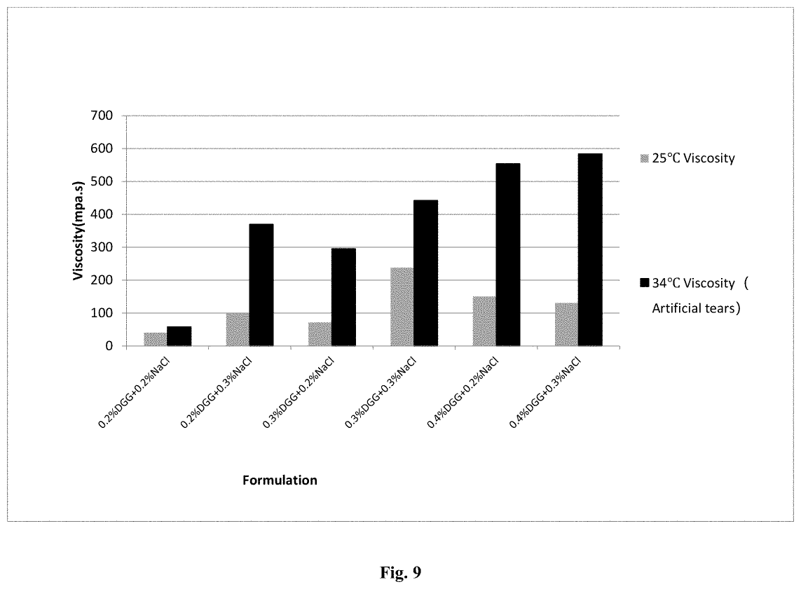

Based on the data shown in Tables 8-11, we have generated histogram charts (see: FIG. 9 to FIG. 12) about the comparative analysis of viscosity changes before and after mixing with artificial tears for samples using different gelling matrix polymers. Comparing the viscosity value at 25° C. and the viscosity value at 34° C. after adding artificial tears indicated that DGG has shown optimal in-situ gel characteristics with the greatest viscosity changes. After adding artificial tears, the viscosity of the formulation greatly increased, and a larger viscosity value was achieved with a small amount of DGG; Xanthan gum, and Carrageenan, and sodium alginate also exhibited certain in-situ gel properties. After adding artificial tears, the viscosity value has also increased to a certain extent, however the viscosity change is not optimal comparing to gellan gum. Therefore, gellen gum is preferred choice as in-situ gelling matrix polymer.

### Example 3: The In-situ Gel of Cyclosporine Micelles in the Present Invention

The formulation of the micellar ophthalmic gel containing 0.05% cyclosporin A is shown as follows:

Cyclosporine A 0.05 wt %, deacetylated gellan gum 0.25 wt %, Polyoxyl 20 Cetostearyl Ether 1.0 wt %, sodium chloride 0.15 wt %, mannitol 3.3 wt %, hydroxyparaben 0.02 wt %, appropriate amount of tromethamine-hydrochloric acid buffer, and injection water were added to make a 100 g ophthalmic gel containing 0.05% cyclosporine micelles(Table 12).

TABLE 12
The composition of example 3 nanomicelle in-situ gel

| Composition | Percentage (wt %) | |
|---|---|---|
| Cyclosporine A | 0.05 | wt % |
| Deacetylated gellan gum | 0.25 | wt % |
| Polyoxyl 20 cetostearyl ether | 1.0 | wt % |
| Sodium chloride | 0.15 | wt % |
| Mannitol | 3.3 | wt % |
| Hydroxyparaben | 0.02 | wt % |

| | |
|---|---|
| Tromethamine hydrochloric acid buffer | As needed |
| Injection water | 100% |

### Sample Preparation

Take a prescribed amount of water for injection into a beaker and stir at a uniform speed with a rotary stirrer. Spread the prescribed amount of deacetylated gellan gum in the above-mentioned water under stirring, and then put it into a 90° C. water bath under stirring for 1 h. The solution was taken out and filtered through 0.45 μm microporous filter membrane while it's hot to get sterilized. Solution 1: precisely weigh the prescribed amount of cyclosporin A, add the prescribed amount of Polyoxyl 20 Cetostearyl Ether to dissolve the cyclosporin A, then add the appropriate amount of sodium chloride, mannitol, hydroxybutyrate, and tromethamine hydrochloric acid buffer respectively. Then pass the solution through a 0.45 μm microporous membrane to obtain Solution 2. Mix Solution 1 and Solution 2 with agitation, and pack into eye drops bottles to obtain cyclosporine nanomicelle in-situ gel.

### Particle Size and Distribution Detection

Measure the particle size and distribution of the 0.05% cyclosporine micelle in-situ gel prepared above using a particle size analyzer. Results are shown in FIG. 9 and Table 13.

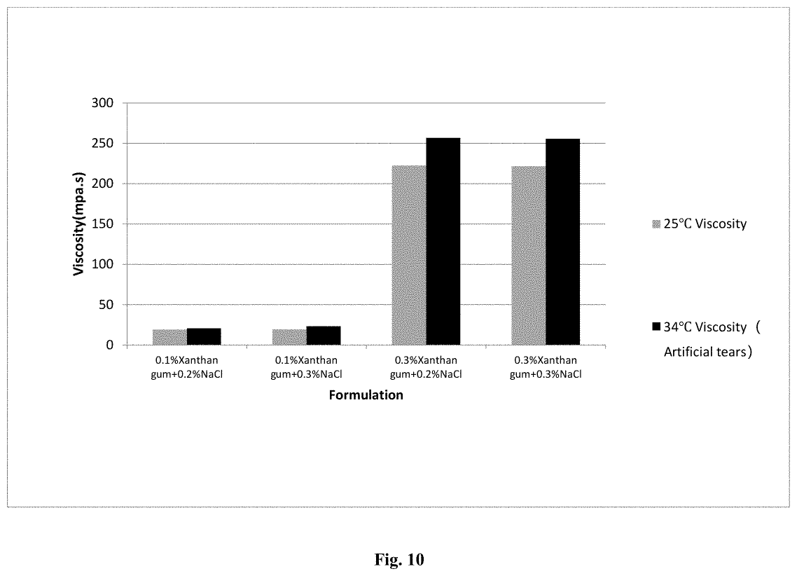

Measure the particle size and distribution of RESTASIS® using a particle size analyzer. Results were shown in FIG. 10 and Table 13.

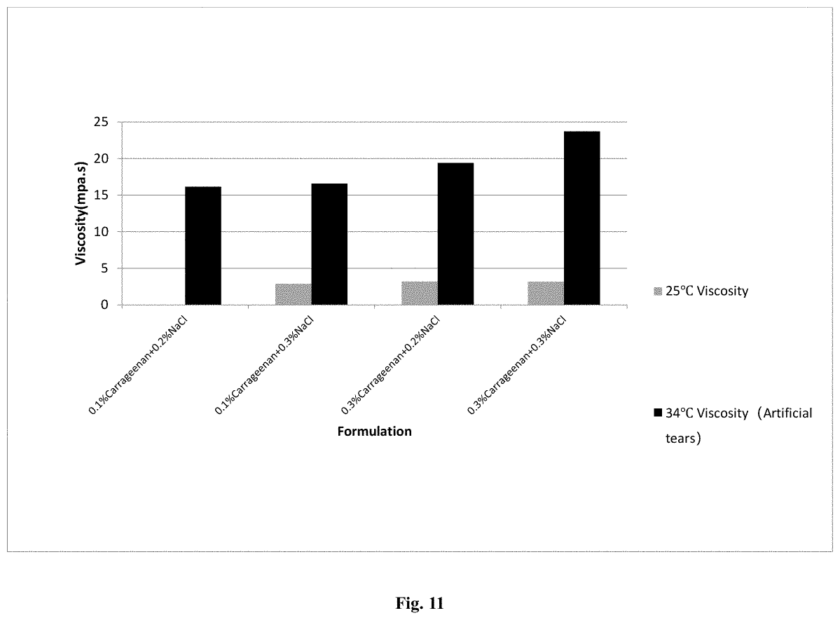

Measure the particle size and distribution of CEQUA® using a particle size analyzer. Results were shown in FIG. 11 and Table 13.

TABLE 13
Comparison of particle sizes of nanomicelles of Example
3 and RESTASIS ® and CEQUA ®

| Sample | Particle size(nm) | PDI |
|---|---|---|
| Example 3 | 12.62 | 0.328 |
| RESTASIS ® | 159.4 | 0.433 |
| CEQUE ® | 22.04 | 0.367 |

From the results in Table 13, it can be seen that the particle size of the nano micelles prepared as sample 3 were smaller than those prepared with Restasis® and Cequa®.

In vitro Release Curve of 0.05% Cyclosporine Micelle Ophthalmic Gel

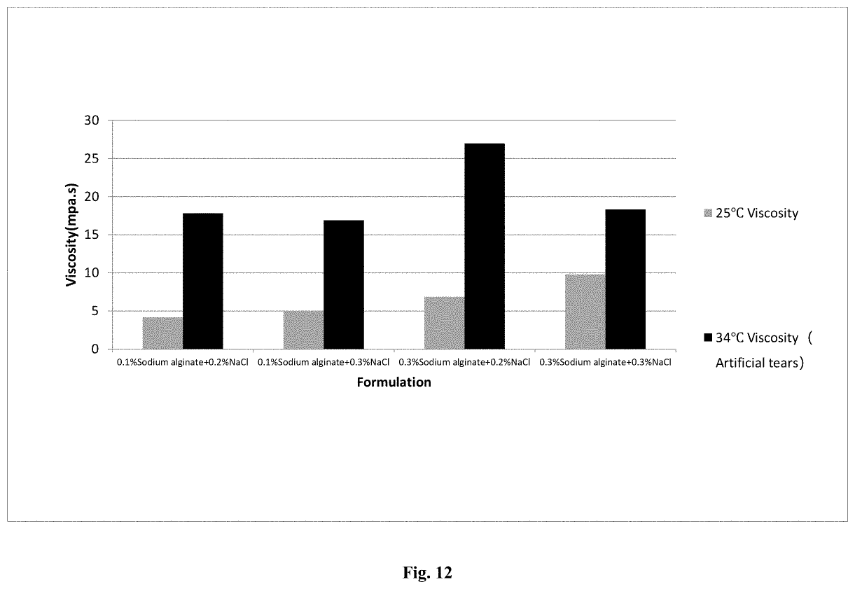

The in vitro release test was carried out by the dissolution method, using 100 mL artificial tears as the medium. The temperature was set at 34±0.5° C. The shaking frequency was 100 r/min. 1 mL of sample was added to the ampoule, then 4 mL of artificial tears was added, and the ampoule was placed into the constant temperature and humidity oscillator. At 0.5, 1, 2, 4, 8, 12, 24, 48 hours, 2 mL of each solution was taken, and 2 mL of fresh medium was added. The sample was filtered through a 0.45 μm microporous membrane filter, and 20 μm of the filtrate was injected into a liquid chromatography system to determine the content (amount) of cyclosporin A. The same method was used to measure the in vitro release profiles of nanomicelles prepared with RESTASIS® and CEQUA®. The release curve was plotted as a percentage of cumulative drug release versus time. We compared the cumulative release data of RESTASIS®, CEQUA® and the sample in Example 3. The release curve was shown in FIG. 12 and Table 14.

TABLE 14
Drug Release Profiles of Example 3 and RESTASIS ® and
CEQUA ®

| | Example 3 | RESTASIS ® | CEQUA ® |
|---|---|---|---|
| Time | (cumulative | (cumulative | (cumulative |
| (h) | release percent) | release percent) | release percent) |
| 0.5 | 0.4% | 80.4% | 46.1% |
| 1 | 10.2% | 90.7% | 87.5% |
| 2 | 25.0% | 91.2% | 91.5% |
| 4 | 36.7% | 90.1% | 91.2% |
| 8 | 55.8% | 90.1% | 91.2% |
| 12 | 66.7% | 90.1% | 91.2% |
| 24 | 75.2% | 90.1% | 91.2% |
| 30 | 88.0% | 90.1% | 91.2% |
| 48 | 95.6% | 90.1% | 91.2% |

The data listed in in FIG. 12 show that the 0.05% cyclosporine micelle ophthalmic gel forming formulation of Example 3 had a significantly sustained release profile than the formulations prepared with RESTASIS® and CEQUA®, as it slowly released 90% of cyclosporine after 30 hours, while formulations of both RESTASIS® and CEQUA® proved to be fast release formulations and released around 90% of cyclosporine within 2 hours. The release rate of the formulation of Example 3 was much slower than the release rates of RESTASIS® and CEQUA®, indicating that the in-situ gel matrix did provide a slow-release profile.

Stability study: 0.05% cyclosporin A micellar ophthalmic gel was prepared and divided into multi-dose eye drop bottles. Samples were stored in a 25° C. stability chamber. Samples were taken on 0, 10, 20 days, 30 days.

Characterization: property, pH, osmotic pressure, viscosity, content, particle size.

TABLE 15
The characterization and stability of the prepared nanomicelle in-situ gel

| | | | | | 34° C. Viscosity | | |
|---|---|---|---|---|---|---|---|
| | | | Osmotic | 25° C. | with Artificial | | |
| | | | pressure | Viscosity | Tears (40:7) | Content | Particle |
| Time | Property | pH | (mOsmol/kg) | (mPa · s) | (mPa · s) | (%) | size (nm) |
| 0 Day | Clear and | 6.86 | 299 | 95.60 | 141.27 | 101.19 | 12.62 |
| | transparent | | | | | | |
| 10 Day | Clear and | 6.61 | 303 | 93.30 | 160.98 | 100.61 | 12.59 |
| | transparent | | | | | | |
| 20 Day | Clear and | 6.58 | 303 | 87.18 | 159.33 | 100.23 | 12.64 |
| | transparent | | | | | | |
| 30 Day | Clear and | 6.56 | 300 | 90.26 | 155.29 | 100.45 | 12.55 |
| | transparent | | | | | | |

### Example 4: The In-situ Gel of Cyclosporine Micelles in the Current Invention

The formulation of the micellar ophthalmic gel containing 0.05% cyclosporin A was shown as followed:

Cyclosporine A 0.05 wt %, DGG 0.3 wt %, HS-15 1.0 wt %, potassium chloride 0.2 wt %, glycerin 0.8 wt %, paraben 0.05%, propyl paraben 0.01%, appropriate amount of phosphate buffer solution, and injection water were added to make a 100 g ophthalmic gel containing 0.05% cyclosporine micelle (Table 16).

TABLE 16
Composition of Example 4 nanomicelle in-situ gel

| Composition | Percentage (wt %) |
|---|---|
| Cyclosporine A | 0.05% |
| Deacetylated gellan gum | 0.3% |
| HS-15 | 1.0% |
| Potassium chloride | 0.2% |
| Glycerin | 0.8% |
| Paraben/propyl paraben | 0.05%/0.01% |
| Phosphate buffer | As needed |
| Injection water | 100% |

### Sample Preparation

Take a prescribed amount of water for injection into a beaker and stir at a uniform speed with a rotary stirrer. Spread the prescribed amount of DGG in the above-mentioned water under stirring, and then put it into a 90° C. water bath under stirring for 1h. The solution was taken out and filtered through 0.45 μm microporous filter membrane while hot to get sterilized Solution 1. Precisely weigh the prescribed amount of cyclosporin A, add the prescribed amount of HS-15 to dissolve the cyclosporin A, add the prescribed amount of potassium chloride, glycerin, paraben, propyl paraben, and phosphate buffer. Then the solution was passed through a 0.45 μm microporous filter to obtain Solution 2. Mix Solution 1 and Solution 2 with agitation, and pack into eye drops bottles to obtain cyclosporine micelle ophthalmic gel.

### Particle Size and Distribution Detection

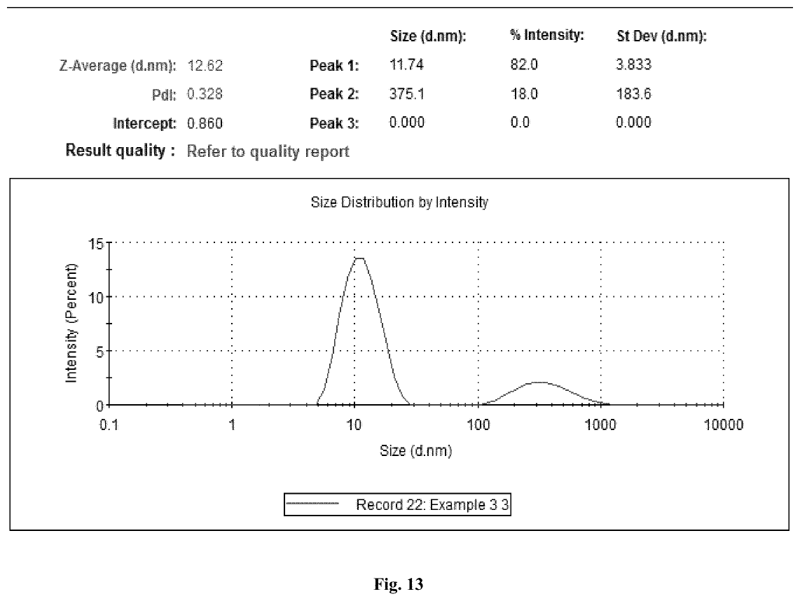

Measure the particle size and distribution of the 0.05% cyclosporine micelle in-situ gel prepared above using a particle size analyzer. Results are shown in FIG. 13 and Table 17.

TABLE 17
Comparison of particle size of Example 4 nanomicelle
with RESTASIS ® and CEQUA ®

| Sample | Particle size (nm) | PDI |
|---|---|---|
| Example 4 | 13.25 | 0.111 |
| RESTASIS ® | 159.4 | 0.433 |
| CEQUE ® | 22.04 | 0.367 |

From the results in Table 19, it can be seen that the particle size is much smaller than that of RESTASIS® and CEQUA®.

In vitro release evaluation: The in vitro release of 0.05% cyclosporine micelle ophthalmic gel was tested.

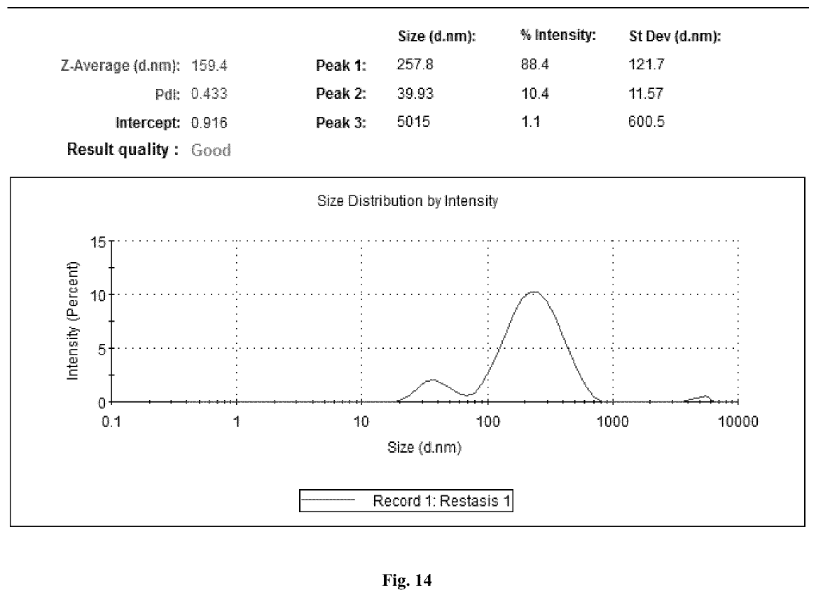

The in vitro release test was carried out by the dissolution method, using 100 ml artificial tears as the medium. The temperature was set at 34±0.5° C. The shaking frequency was 100 r/min. 1 mL of sample was added to the ampoule, then 4 mL of artificial tears was added, and the ampoule was placed into the constant temperature and humidity oscillator; at 0.5, 1, 2, 4, 8, 12, 24, 48 hours 2 ml of each solution was taken, and 2 mL of fresh medium was added. The sample was filtered through a 0.45 μm membrane filter, and 20 μL was injected into the liquid chromatography system to determine the content of cyclosporin A. The release curve was plotted as a percentage of cumulative drug release versus time. We compared the cumulative release data of RESTASIS®, CEQUA® and the sample in Example 4. The release curve was shown in FIG. 14 and Table 18.

TABLE 18
Drug release of example 4 and RESTASIS ® and CEQUA ®

| | Example 4 | RESTASIS ® | CEQUE ® |
|---|---|---|---|
| Time | (cumulative | (cumulative | (cumulative |
| (h) | release percent) | release percent) | release percent) |
| 0.5 | 5.9% | 80.4% | 46.1% |
| 1 | 40.9% | 90.7% | 87.5% |
| 2 | 60.2% | 91.2% | 91.5% |
| 4 | 70.0% | 91.2% | 91.5% |
| 8 | 74.9% | 91.2% | 91.5% |
| 12 | 78.6% | 91.2% | 91.5% |
| 24 | 80.8% | 91.2% | 91.5% |
| 30 | 82.6% | 91.2% | 91.5% |
| 48 | 92.4% | 91.2% | 91.5% |

From the results shown in FIG. 14, it can be seen that the 0.05% cyclosporine micelle ophthalmic gel forming formulation Example 4 comparing to RESTASIS® and CEQUA® has shown a significant sustained release profile and slowly release 90% of cyclosporine after 30 hours, while both RESTASIS® and CEQUA® proved to be fast release formulations and release around 90% of cyclosporine within 2 hours. The release rate is much slower than the release rate of RESTASIS® and CEQUA®, indicating that the in-situ gel matrix provided a slow-release profile.

Stability study: 0.05% cyclosporin A micellar ophthalmic gel was prepared and divided into multi-dose eye drop bottles. The bottles were stored in a 25° C. stability Chamber. Samples were taken on 0, 10, 20 days, 30 days.

Characterization: Appearance, pH, osmotic pressure, viscosity, content, particle size. The experimental results are shown in Table 19 below.

TABLE 19
Characterization and Stability of Prepared Nanomicelle In-Situ Gel

| | | | | | 34° C. Viscosity | | |
|---|---|---|---|---|---|---|---|
| | | | Osmotic | 25° C. | with Artificial | | |
| Time | | | pressure | Viscosity | Tears (40:7) | Content | Particle |
| (Day) | Appearance | pH | (mOsmol/kg) | (mpa · s) | (mpa · s) | (%) | size (nm) |
| 0 | Clear and | 6.84 | 297 | 82.37 | 151.88 | 99.68 | 13.04 |
| | transparent | | | | | | |
| 10 | Clear and | 6.73 | 300 | 77.94 | 156.86 | 99.56 | 13.22 |
| | transparent | | | | | | |
| 20 | Clear and | 6.71 | 302 | 73.80 | 163.84 | 99.48 | 13.24 |
| | transparent | | | | | | |
| 30 | Clear and | 6.69 | 298 | 76.55 | 159.72 | 99.15 | 13.28 |
| | transparent | | | | | | |

### Example 5: In-situ Gel with Cyclosporine Micelles

The specific prescription of the micellar ophthalmic gel containing 0.05% cyclosporin A was shown as follows:

Cyclosporine A 0.05 wt %, deacetylated gellan gum 0.4 wt %, Soluplus 0.9 wt %, calcium chloride 0.2 wt %, propylene glycol 0.8 wt %, potassium sorbate 0.01 wt %, appropriate amount of borate buffer, and water for injection were added to make a 100 g of ophthalmic gel containing 0.05% cyclosporine micelles (see Table 20).

TABLE 20
The composition of nanomicelle-containing
in-situ gel in Example 5

| Composition | Percentage(wt %) |
|---|---|
| Cyclosporine A | 0.05% |
| Deacetylated gellan gum | 0.4% |
| Soluplus | 0.9% |
| Calcium chloride | 0.2% |
| Propylene glycol | 0.8% |
| Potassium sorbate | 0.01% |
| Borate buffer | As needed |
| Injection water | 100% |

### Sample Preparation

Soluplus in a prescribed amount was weighted into a 250 mL beaker. 10 mL of absolute ethanol was added to dissolve prescribed amount of cyclosporin A. The solution was heated at 80° C. to evaporate ethanol, and colorless and transparent film was obtained. 20 ml of deionized water was added to hydrate the film for 15 hours to make Solution 1. Propylene glycol, calcium chloride, potassium sorbate, deacetylated gellan gum were weighted according to the prescribed amounts, and added into 70 ml of deionized water, heated at 90° C. for 1 hour under stirring until gellan gum was completely dissolved. Solution 2 was obtained after cooling. Solution 2 was slowly added into Solution 1 under stirring, and finally the pH was adjusted with borate buffer. Deionized water was added to make the final weight of 100 g. Samples were filtered through 0.22 μm microporous membrane filter for sterilization.

### Particle Size and Distribution Detection

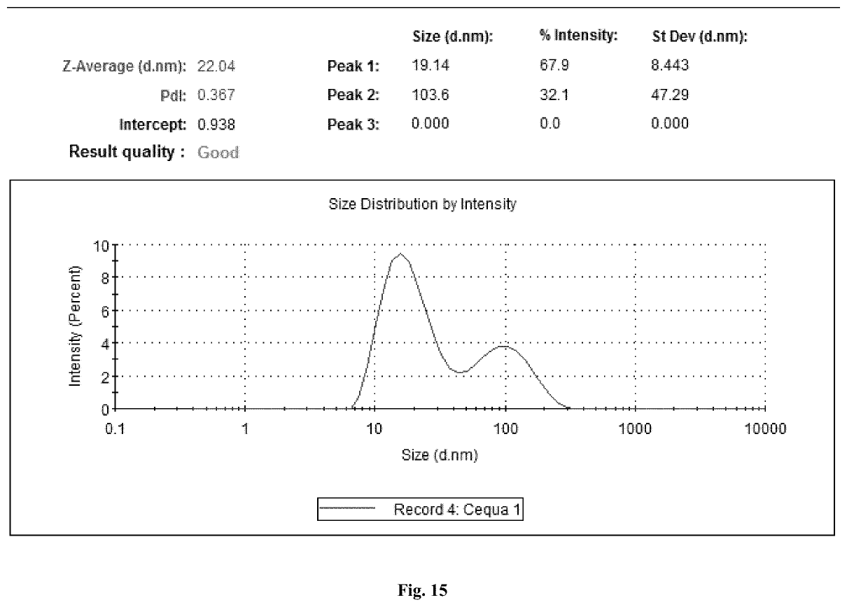

Measure the particle size and distribution of the 0.05% cyclosporine micelle in-situ gel prepared above using a particle sizer. Results are shown in FIG. 15 and Table 21.

TABLE 21
Comparison of particle size of Example 5 nanomicelle
with RESTASIS ® and CEQUA ®

| Sample | Particle size(nm) | PDI |
|---|---|---|
| Example 5 | 71.93 | 0.125 |
| RESTASIS ® | 159.4 | 0.433 |
| CEQUA ® | 22.04 | 0.367 |

The results in Table 21 and FIG. 15 show that the micellar particle size of Example 5 was much smaller than that of RESTASIS® but bigger than CEQUA®.

In vitro release evaluation: The in vitro release curve of 0.05% cyclosporine micelle ophthalmic gel was generated.

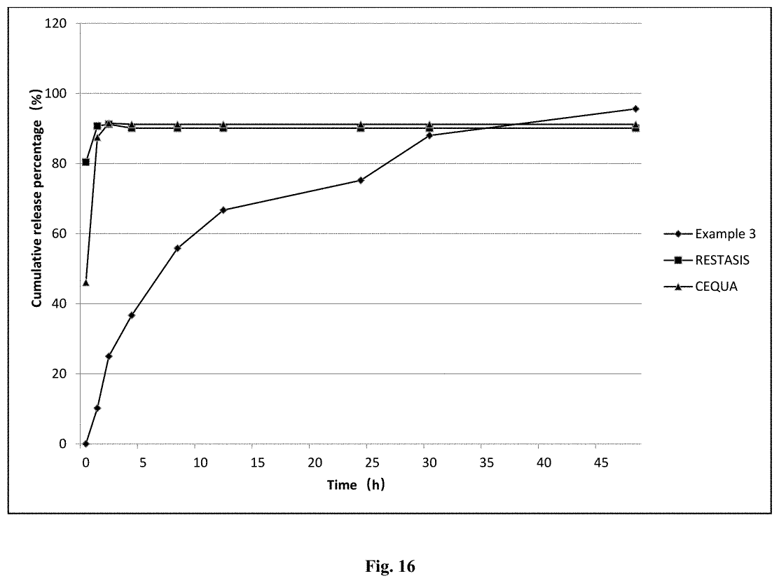

The in vitro release test was carried out by the dissolution method, using 100 ml artificial tears as the medium. The temperature was set at 34±0.5° C. The shaking frequency was 100 r/min. 1ml of sample was added to the ampoule, then 4m1 of artificial tears was added, and the ampoule was placed into the constant temperature and humidity oscillator; at 0.5, 1, 2, 4, 8, 12, 24, 48 hours, 2 mL of each solution was taken and 2 ml of fresh medium was added. The sample was filtered through a 0.45 μm microporous membrane filter, and 20 μL was injected into the liquid chromatography system to determine the content of cyclosporin A. The release curve was plotted as a percentage of cumulative drug release versus time. We compared the cumulative release data of RESTASIS®, CEQUA® and the sample in Example 5. The release curve was shown in FIG. 16 and Table 22.

TABLE 22
Drug release of example 5 and RESTASIS ® and CEQUA ®

| | Example 5 | RESTASIS ® | CEQUA ® |
|---|---|---|---|
| Time | (cumulative | (cumulative | (cumulative |
| (h) | release percent) | release percent) | release percent) |
| 0.5 | 10.3% | 80.4% | 46.1% |
| 1 | 20.2% | 90.7% | 87.5% |
| 2 | 29.3% | 91.2% | 91.5% |
| 4 | 36.8% | 91.2% | 91.5% |
| 8 | 44.2% | 91.2% | 91.5% |
| 12 | 60.3% | 91.2% | 91.5% |
| 24 | 81.5% | 91.2% | 91.5% |
| 30 | 86.7% | 91.2% | 91.5% |
| 48 | 93.1% | 91.2% | 91.5% |

It can be seen from the results in FIG. 16 that the 0.05% cyclosporine micelle ophthalmic gel forming formulation Example 5 comparing to RESTASIS® and CEQUA® has shown a significant sustained release profile and slowly release 90% of cyclosporine after 30 hours, while both RESTASIS® and CEQUA® proved to be fast release formulations and release around 90% of cyclosprine within 2 hours. The release rate is much slower than the release rate of RESTASIS® and CEQUA®, indicating that the in-situ gel matrix provided a slow-release profile.

Stability study: 0.05% cyclosporin A micellar ophthalmic gel was prepared and divided into multi-dose eye drop bottles. Samples were stored in a 25° C. stability chamber. Samples were taken on 0, 10, 20 days, 30 days.

Characterization: appearance, pH, osmotic pressure, viscosity, content, particle size.

Experimental results (Table 23):

TABLE 23
The characterization and stability of the prepared nanomicelle in-situ gel

| | | | | | 34° C. Viscosity | | |
|---|---|---|---|---|---|---|---|
| | | | Osmotic | 25° C. | With Artificial | | |
| Time | | | pressure | Viscosity | Tears(40:7) | Content | Particle |
| (Day) | Appearance | pH | (mOsmol/kg) | (mpa · s) | (mpa · s) | (%) | size (nm) |
| 0 | Milky white | 7.59 | 299 | 70.76 | 184.23 | 99.78 | 71.93 |
| 10 | Milky white | 7.44 | 300 | 67.99 | 183.59 | 98.83 | 71.36 |
| 20 | Milky white | 7.35 | 298 | 61.83 | 206.33 | 98.59 | 72.89 |
| 30 | Milky white | 7.28 | 301 | 68.29 | 198.55 | 98.66 | 71.43 |

### Example 6: The In-situ Gel of Cyclosporine Micelles in the Current Invention

The formulation of the micellar ophthalmic gel containing 0.05% cyclosporin A is shown as follows:

Cyclosporine A 0.05 wt %, DGG 0.3 wt %, HS-15 0.25 wt %, RH-40 1.0 wt %, sodium chloride 0.25 wt %, mannitol 3.3 wt %, paraben fat 0.05%, Propylparaben 0.01 wt %, appropriate amount of tromethamine hydrochloric acid buffer solution, and water for injection were added to make a 100 g of ophthalmic gel containing 0.05% cyclosporine micelles (Table 24).

TABLE 24
The composition of example 6 nanomicelle in-situ gel

| Composition | Percentage(wt %) |
|---|---|
| Cyclosporine A | 0.05 w % |
| Deacetylated gellan gum | 0.3 w % |
| HS-15/RH-40 | 0.25 w %/1.0 t % |
| Sodium chloride | 0.25% |
| Mannitol | 3.3% |
| Paraben fat/Propylparaben | 0.05%/0.01% |
| Tromethamine hydrochloric acid buffer | As needed |
| Injection water | 100% |

### Sample Preparation

Take a prescribed amount of water for injection into a beaker and stir at a uniform speed with a rotary stirrer. Spread the prescribed amount of deacetylated gellan gum in the above-mentioned water under stirring, and then put it into a 90° C. water bath under stirring for 1 hour. The solution was taken out and filtered through 0.45 μm microporous filter membrane while hot to get sterilized Solution 1. Precisely weigh the prescribed amount of cyclosporin A, add the prescribed amounts of HS-15 and RH-40 to dissolve the cyclosporin A, Add the appropriate amount of sodium chloride, mannitol, paraben, propyl paraben, and tromethamine hydrochloride buffer. Then the solution was passed through a 0.45 μm microporous membrane filter to obtain Solution 2. Mix Solution 1 and Solution 2 with agitation to obtain cyclosporine micelle ophthalmic gel and pack into eye drops bottles.

### Particle Size and Distribution Measurement

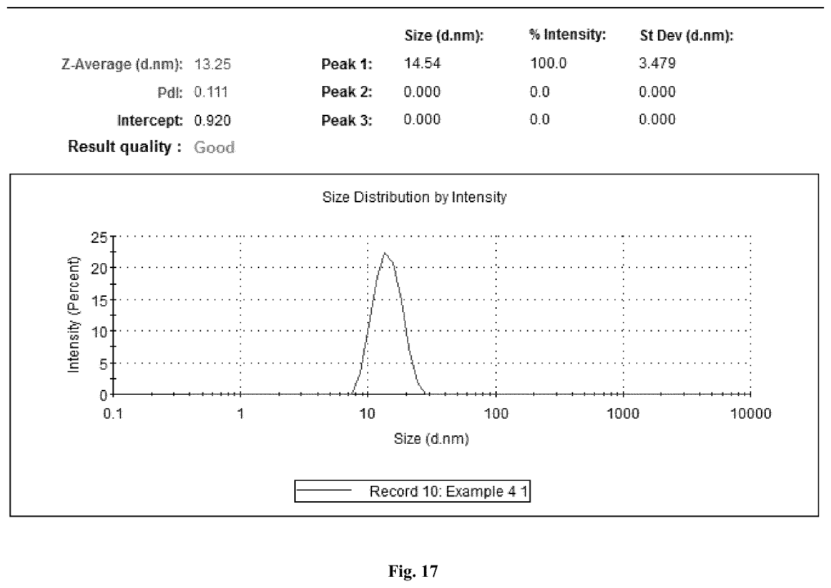

The particle size and distribution index of the 0.05% cyclosporine micelles-containing in-situ gel prepared above was measure using a particle size analyzer, and the results are listed below in FIG. 17 and Table 25.

TABLE 25
Comparison of particle size of nanomicelles in Example 6
and RESTASIS ® and CEQUA ®

| Sample | Particle size(nm) | PDI |
|---|---|---|
| Example 6 | 14.57 | 0.168 |
| RESTASIS ® | 159.4 | 0.433 |
| CEQUE ® | 22.04 | 0.367 |

From the results in Table 25, it can be seen that the particle size is much smaller than that of RESTASIS® and CEQUA®.

In vitro release evaluation: The in vitro release curve of 0.05% cyclosporine micelle ophthalmic gel was generated.

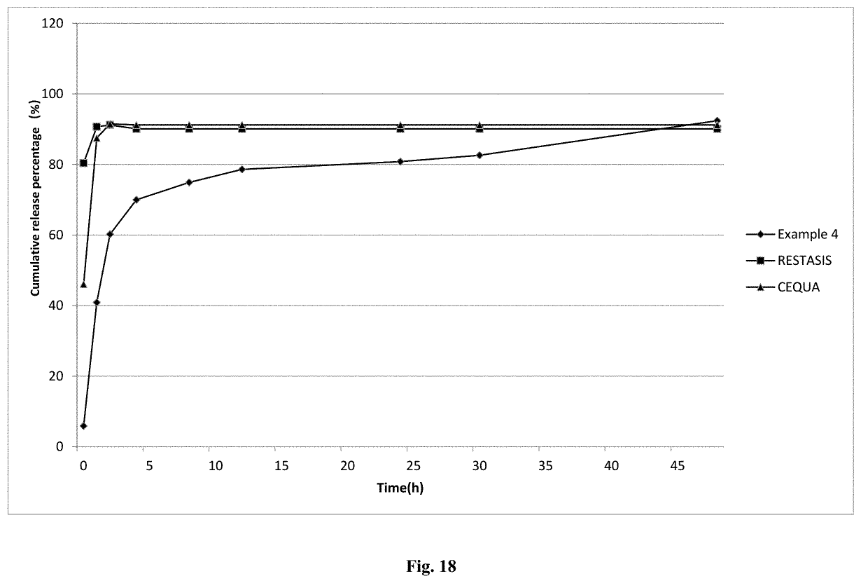

The in vitro release test was carried out by the dissolution method, using 100 ml artificial tears as the medium. The temperature was set at 34±0.5° C. The shaking frequency was 100 r/min. 1 ml of sample was added to the ampoule, then 4 ml of artificial tears was added, and the ampoule was placed into the constant temperature and humidity oscillator; at 0.5, 1, 2, 4, 8, 12, 24, 48 hours, 2 ml of each solution was taken, and 2 ml of fresh medium was added. The sample was filtered through a 0.45 μm microporous membrane filter, and 20 μL was injected into the liquid chromatography system to determine the content of cyclosporine A. The release curve was plotted as a percentage of cumulative drug release versus time. We compared the cumulative release data of RESTASIS®, CEQUA® and the sample in Example 5.The release curve was shown in FIG. 18 and Table 26.

TABLE 26
Drug release of example 6 and RESTASIS ® and CEQUA ®

| | Example 6 | RESTASIS ® | CEQUA ® |
|---|---|---|---|
| Time | (cumulative | (cumulative | (cumulative |
| (h) | release percent) | release percent) | release percent) |
| 0.5 | 6.1% | 80.4% | 46.1% |
| 1 | 29.8% | 90.7% | 87.5% |
| 2 | 43.0% | 91.2% | 91.5% |
| 4 | 55.2% | 91.2% | 91.5% |
| 8 | 67.6% | 91.2% | 91.5% |
| 12 | 72.8% | 91.2% | 91.5% |
| 24 | 81.5% | 91.2% | 91.5% |
| 30 | 84.5% | 91.2% | 91.5% |
| 48 | 91.6% | 91.2% | 91.5% |

It can be seen from the results in FIG. 18 that the 0.05% cyclosporine micelle ophthalmic gel forming formulation Example 6 comparing to RESTASIS® and CEQUA® has shown a significant sustained release profile and slowly release 90% of cyclosporine after 30 hours, while both RESTASIS® and CEQUA® proved to be fast release formulations and release around 90% of cyclosprine within 2 hours. The release rate is much slower than the release rate of RESTASIS® and CEQUA®, indicating that the in-situ gel matrix provided a slow-release profile.

Stability study: 0.05% cyclosporin A micellar ophthalmic gel was prepared and divide it into multi-dose eye drop bottles. Samples were stored in a 25° C. stability chamber. Samples were taken on 0, 10, 20 days, 30 days.

Characterization: Appearance, pH, osmotic pressure, viscosity, content, particle size. Experimental results are listed in Table 27 below.

TABLE 27
The characterization and stability of the prepared nanomicelle in-situ gel

| | | | | | 34° C. Viscosity | | |
|---|---|---|---|---|---|---|---|
| | | | Osmotic | 25° C. | with Artificial | | |
| Time | | | pressure | Viscosity | Tears (40:7) | Content | Particle |
| (Day) | Appearance | pH | (mOsmol/kg) | (mpa · s) | (mpa · s) | (%) | size (nm) |
| 0 | Clear and | 6.91 | 308 | 76.25 | 247.82 | 99.01% | 14.57 |
| | transparent | | | | | | |
| 10 | Clear and | 6.85 | 304 | 61.91 | 257.50 | 97.13 | 15.25 |
| | transparent | | | | | | |
| 20 | Clear and | 6.74 | 309 | 60.77 | 241.18 | 98.11 | 15.85 |
| | transparent | | | | | | |
| 30 | Clear and | 6.69 | 305 | 66.97 | 239.25 | 98.65 | 14.99 |
| | transparent | | | | | | |

### Example 7: The In-situ Gel of Cyclosporine Micelles in the Current Invention

The formulation of the micellar ophthalmic gel containing 0.09% cyclosporin A is shown as follows:

Cyclosporine A 0.09 wt %, DGG 0.3 wt %, HS-15 0.25 wt %, RH-40 1.0 wt %, sodium chloride 0.25 wt %, mannitol 3.3 wt %, paraben fat 0.05% ,propylparaben 0.01 wt %, appropriate amount of tromethamine hydrochloric acid buffer solution, and injection water were added to make a 100 g of ophthalmic gel containing 0.05% cyclosporine micelles (Table 28).

TABLE 28
Composition of Example 7 nanomicelle in-situ gel

| Composition | Percentage(wt %) |
|---|---|
| Cyclosporine A | 0.09% |
| Deacetylated gellan gum | 0.3% |
| HS-15/RH-40 | 0.25%/1.0% |
| Sodium chloride | 0.25% |
| Mannitol | 3.3% |
| Paraben fat/Propylparabe | 0.05%/0.01% |
| Tromethamine hydrochloric acid buffer | As needed |
| Injection water | 100% |

### Sample Preparation

Take a prescribed amount of water for injection into a beaker and stir at a uniform speed with a rotary stirrer. Spread the prescribed amount of deacetylated gellan gum in the above-mentioned water under stirring, and then put it into a 90° C. water bath under stirring for 1 hour. The solution was taken out and filtered through 0.45 μm microporous membrane filter while hot to get sterilized Solution 1. Precisely weigh the prescribed amount of cyclosporin A, add the prescribed amounts of HS-15 and RH-40 to dissolve the cyclosporin A, Add the appropriate amount of sodium chloride, mannitol, paraben, propyl paraben, and tromethamine hydrochloride buffer. Then the solution was passed through a 0.45 μm microporous membrane filter to obtain Solution 2. Mix Solution 1 and Solution 2 with agitation to obtain cyclosporine micelle ophthalmic gel, and pack into eye drops bottles.

### Particle Size and Distribution Measurement

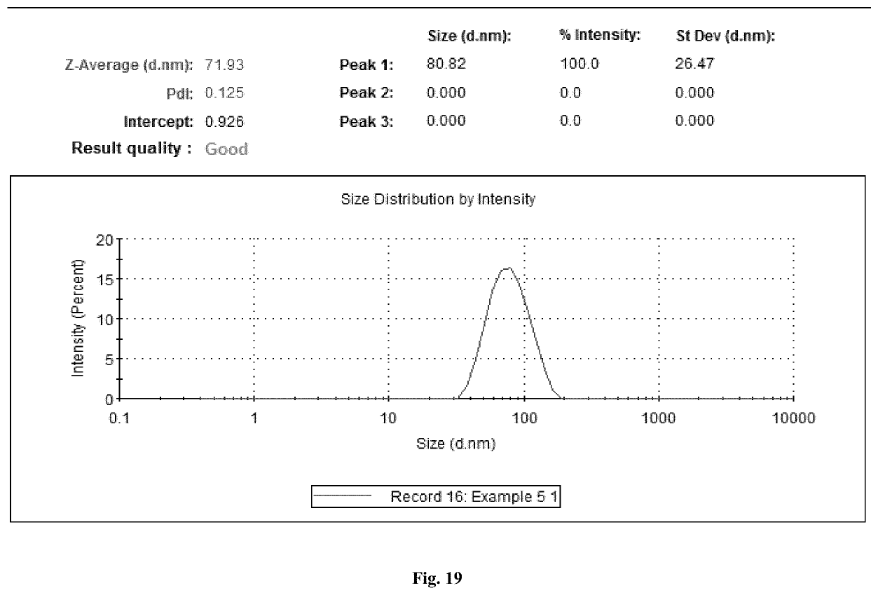

Measure the particle size and distribution of the 0.09% cyclosporine micelle in-situ gel prepared above using a particle size analyzer. Results were shown in FIG. 19 and Table 29.

TABLE 29
Comparison of particle size of nanomicelles in Example 7
and RESTASIS ® and CEQUA ®

| Sample | Particle size(nm) | PDI |
|---|---|---|
| Example 7 | 14.10 | 0.097 |
| RESTASIS ® | 159.4 | 0.433 |
| CEQUE ® | 22.04 | 0.367 |

The results in Table 29 show that the particle size of nanomicelles in Example 7 was smaller than that of RESTASIS® and CEQUA®.

In vitro release evaluation: The in vitro release curve of 0.09% cyclosporine micelle ophthalmic gel was tested.

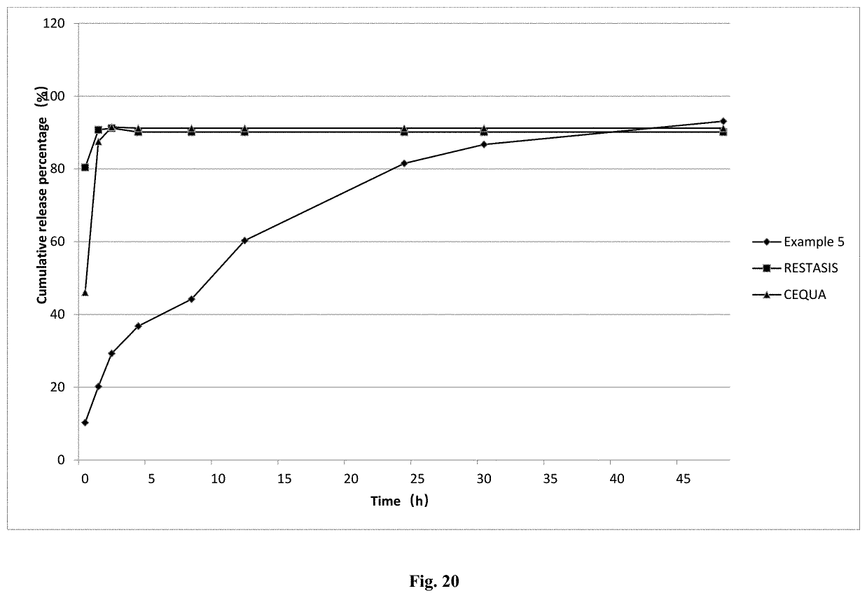

The in vitro release test was carried out by the dissolution method, using 100 ml artificial tears as the medium. The temperature was set at 34±0.5° C. The shaking frequency was 100 r/min. 1 ml of sample was added to the ampoule, then 4 ml of artificial tears was added, and the ampoule was placed into the constant temperature and humidity oscillator; at 0.5, 1, 2, 4, 8, 12, 24, 48 hours, 2 ml of each solution was taken, and 2ml of fresh medium was added. The sample was filtered through a 0.45 μm microporous membrane filter, and 20 μL was injected into the liquid chromatography system to determine the content of cyclosporin A. The release curve was plotted as a percentage of cumulative drug release versus time. We compared the cumulative release data of RESTASIS®, CEQUA® and the sample in Example 5. The release curve was shown in FIG. 20 and Table 30.

TABLE 30
Drug release of Example 7 and RESTASIS ® and CEQUA ®

| | Example 7 | RESTASIS ® | CEQUA ® |
|---|---|---|---|
| Time | (cumulative | (cumulative | (cumulative |
| (h) | release percent) | release percent) | release percent) |
| 0.5 | 6.57% | 80.4% | 46.1% |
| 1 | 26.6% | 90.7% | 87.5% |
| 2 | 37.9% | 91.2% | 91.5% |
| 4 | 60.5% | 91.2% | 91.5% |
| 8 | 69.3% | 91.2% | 91.5% |
| 12 | 75.8% | 91.2% | 91.5% |
| 24 | 86.9% | 91.2% | 91.5% |
| 30 | 89.6% | 91.2% | 91.5% |
| 48 | 92.6% | 91.2% | 91.5% |

From the results in FIG. 20, it can be seen that the 0.05% cyclosporine micelle ophthalmic gel forming formulation Example 7 comparing to RESTASIS® and CEQUA® has shown a significant sustained release profile and slowly release 90% of cyclosporine after 30 hours, while both RESTASIS® and CEQUA® proved to be fast release formulations and release around 90% of cyclosprine within 2 hours. The release rate is much slower than the release rate of RESTASIS® and CEQUA®, indicating that the in-situ gel matrix provided a slow-release profile.

Stability study: 0.09% cyclosporin A micellar ophthalmic gel was prepared and divide it into multi-dose eye drop bottles. Samples were stored in a 25° C. stability chamber. Samples were taken on 0, 10, 20 days, 30 days.

Characterization: appearance, pH, osmotic pressure, viscosity, content, particle size. The results are listed in Table 31 below.

TABLE 31
Characterization and stability of nanomicelle-containing in-situ gel

| | | | | | 34° C. Viscosity | | |
|---|---|---|---|---|---|---|---|
| | | | Osmotic | 25° C. | with artificial | | |
| Time | | | pressure | Viscosity | Tears (40:7) | Content | Particle |
| (Day) | Property | pH | (mOsmol/kg) | (mpa · s) | (mpa · s) | (%) | size (nm) |
| | Clear and | 6.86 | 291 | 83.79 | 166.56 | 98.37 | 14.10 |
| | transparent | | | | | | |
| 10 | Clear and | 6.79 | 295 | 79.60 | 167.03 | 98.33 | 14.31 |
| | transparent | | | | | | |
| 20 | Clear and | 6.76 | 293 | 80.55 | 172.66 | 98.26 | 14.26 |
| | transparent | | | | | | |
| 30 | Clear and | 6.75 | 293 | 82.41 | 169.37 | 98.05 | 14.08 |
| | transparent | | | | | | |

### Example 8: In Vitro Dialysis Release Test

In vitro dialysis release test was conducted on Samples 1-6, RESTASIS®, and CEQUA®. The formulations/compositions of tested Samples 1-6 are listed below in Table 32.

TABLE 32
Compositions of the nanomicelles samples tested for dialysis release

| | Sample 1 | Sample 2 | Sample 3 | Sample 4 | Sample 5 | Sample 6 |
|---|---|---|---|---|---|---|
| Cyclosporine | 0.03% | 0.05% | 0.09% | 0.03% | 0.05% | 0.09% |
| Polyoxyl 20 | 0.6% | 0.6% | 1.0% | — | — | — |
| Cetostearyl | | | | | | |
| Ether | | | | | | |
| Polyoxyl 15 | — | — | — | 0.25% | 0.25% | 0.25% |
| Hydroxystearate | | | | | | |
| Polyoxyethylene | — | — | — | 1.0% | 1.0% | 1.0% |
| 40 castor oil | | | | | | |
| Mannitol | 3.3% | 3.3% | 3.3% | 3.3% | 3.3% | 3.3% |
| Water for | Up to 100 g | Up to 100 g | Up to 100 g | Up to 100 g | Up to 100 g | Up to 100 g |
| Injection | | | | | | |

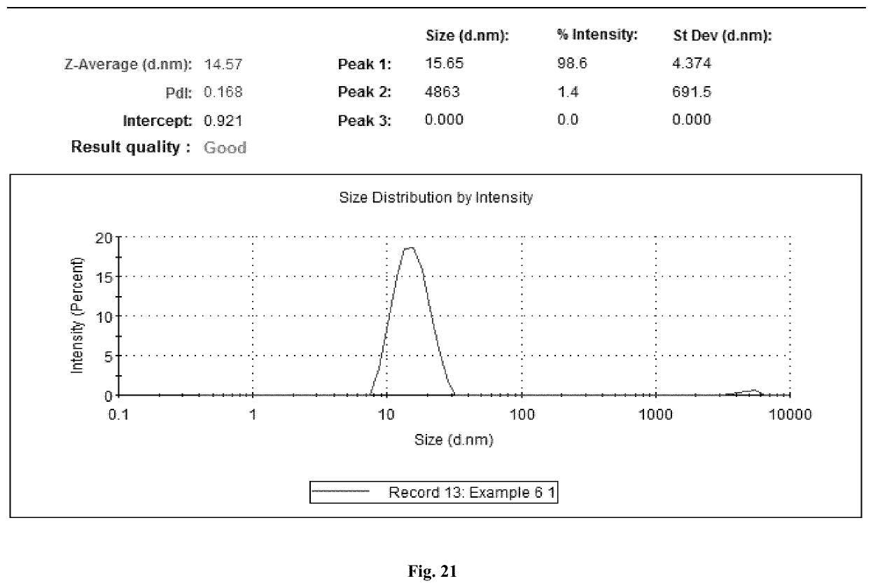

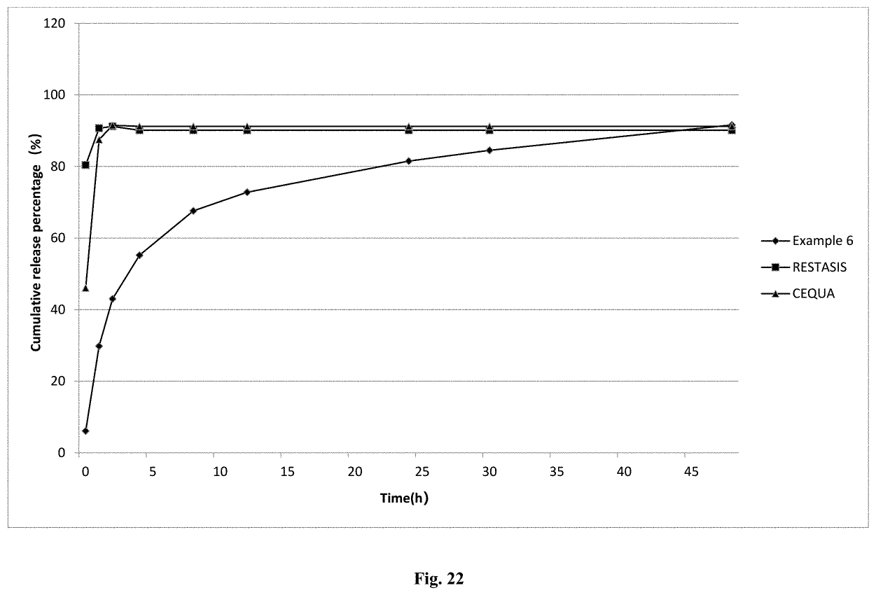

2 mL of each of Samples 1-6, RESTASIS® and CEQUA® was taken and added to a 14 KDa dialysis bag, which was then put into 200 mL artificial tear (containing 30% ethanol) pre-warmed to 34.5° C. The sample was shaken in water bath shaker at 100 rpm, and , take out 5 ml release medium at certain time point (0.5, 1, 2, 4, 6, 8, 12, 18 h), and add same volume of release medium (pre-warm to 34.5° C.) quickly. The available cyclosporine concentration was determined using HPLC. The release curve is obtained by plotting the cumulative release percentage of the drug against time. We compared the cumulative release data of RESTASIS®, CEQUA® and Sample 1-3. The release curve is shown in Table 33 and FIG. 21. Additionally, we compared the cumulative release data of RESTASIS®, CEQUA® and the Sample 4-6. The release curve is shown in Table 33 and FIG. 22.

TABLE 33
Comparison of drug release from samples 1-6 and RESTASIS ® and CEQUA ®

| | Sample1 | Sample2 | Sample3 | Sample4 | Sample5 | Sample6 | RESTASIS ® | CEQUA ® |
|---|---|---|---|---|---|---|---|---|
| Time(h) | Cumulative release (μg/mL) | | | | | | | |
| 0.5 | 0.515 | 0.669 | 0.433 | 0.209 | 0.291 | 0.294 | 0.304 | 0.286 |
| 1 | 0.656 | 0.847 | 0.601 | 0.348 | 0.513 | 0.842 | 0.403 | 0.877 |
| 2 | 0.809 | 0.952 | 1.389 | 0.622 | 0.846 | 1.741 | 0.570 | 1.504 |
| 4 | 1.125 | 1.542 | 3.612 | 1.035 | 1.384 | 3.208 | 0.911 | 2.896 |
| 8 | 1.756 | 2.028 | 5.694 | 1.514 | 1.976 | 5.247 | 1.566 | 4.211 |
| 12 | 2.138 | 2.869 | 7.553 | 1.966 | 2.625 | 7.189 | 1.825 | 6.189 |
| 18 | 2.612 | 4.125 | 8.972 | 2.374 | 3.975 | 8.687 | 2.183 | 7.569 |

Polyoxyl 20 cetostearyl ether was used as a solubilizer to prepare cyclosporine Sample 1 (0.03 % CsA), Sample 2 (0.05 % CsA) and Sample 3 (0.09 % CsA). The drug permeation from those samples was compared with that of RESTASIS® (0.05 % CsA) and CEQUA® (0.09 % CsA) using the semipermeable membrane as shown in FIG. 22. The cumulative release of Sample 2 (0.05% CsA) was significantly higher than that of RESTASIS® (0.05 % CsA) and the cumulative release of Sample 3 (0.09 % CsA) was significantly higher than that of CEQUA® (0.09 % CsA). The cumulative release of Sample 1 (0.03 % CsA) was similar to that of RESTASIS® (0.05 % CsA). The results demonstrated that in the simulated corneal penetration test using semi-permeable membrane, a smaller micelle particle size significantly increased the penetration of cyclosporine in the cornea and thus reduced the concentration of the drug in the ophthalmic preparation to achieve the same or even better therapeutic effect. This is a surprising discovery that potentially we can use less concentration of cyclosporine to achieve similar therapeutic effect with the smaller particle size nanomicelle formulation, and we can expect our formulation with same concentrations as RESTASIS® (0.05 % CsA) or CEQUA® (0.09 % CsA) can achieve much better therapeutic effect. In addition, reducing the concentration of the drug will also reduce the irritation of the drug to the eyes.

Polyoxyl 15 Hydroxystearate and Polyoxyethylene 40 Castor oil were used as solubilizers to prepare Sample 4 (0.03 % CsA), Sample 5 (0.05 % CsA) and Sample 6 (0.09 % CsA). The drug permeation from those Samples was compared with that of RESTASIS® (0.05% CsA) and CEQUA® (0.09 % CsA) using the semipermeable membrane as shown in FIG. 22. While the cumulative release of Sample 4 (0.03 % CsA) was similar to that of RESTASIS® (0.05 % CsA), the cumulative release of Sample 5 (0.05 % CsA) was significantly higher than that of RESTASIS® (0.05 % CsA) and the cumulative release of Sample 6 (0.09 % CsA) was significantly higher than that of CEQUA® (0.09 % CsA). This further confirmed that smaller micelle particle size greatly increased the penetration of cyclosporine in the cornea and further reduced the need for higher concentration of the drug in the ophthalmic preparation to achieve the same or even better therapeutic effect. These advantages may also help reduce the frequency of drug administration as well.

1. An aqueous ophthalmic formulation comprising cyclosporine A, a solubilizer, an osmotic pressure regulator, a pH regulator, a viscosity adjuster, and water, wherein micelles with particle size no greater than 20 nm are formed with cyclosporine and the solubilizer and contained in the formulation.

2. The aqueous ophthalmic formulation of claim 1, further comprising a gel-forming polysaccharide polymer, wherein a gel is formed in situ at the physiological temperature with instant viscosity increase upon instillation of the formulation into the eye.

3. The aqueous ophthalmic formulation of claim 1, wherein cyclosporine has a concentration of 0.01% to 5% by weight in the formulation.

4. The aqueous ophthalmic formulation of claim 1, wherein the solubilizer comprises Polyoxyl 20 Cetostearyl Ether, Polyoxyl 15 Hydroxystearate, Soluplus, Polyoxyethylene hydrogenated castor oil, Polyoxyethylene castor oil, Vitamin E Polyethylene Glycol Succinate, or any combination thereof.

5. The aqueous ophthalmic formulation of claim 1, wherein the solubilizer has a concentration of 0.01% to 10% by weight in the formulation.

6. The aqueous formulation of claim Zany of claim 2, wherein the polysaccharide is contained in the formation at a concentration of 0.1% to 0.6% by weight.

7. The aqueous ophthalmic formulation of claim 2, wherein the polysaccharide comprises deacetylated gellan gum (DGG), xanthan, sodium alginate, carrageenan, or any mixture thereof.

8. The aqueous ophthalmic formulation of claim 2, wherein the polysaccharide comprises deacetylated gellan gum (DGG).

9. The aqueous ophthalmic formulation of claim 1, wherein said osmotic pressure regulator comprises sodium chloride, mannitol, glucose, sorbitol, glycerin, polyethylene glycol, propylene glycol, or any combination thereof.

10. The aqueous ophthalmic formulation of claim 1, wherein the osmotic pressure regulator is in the formulation at a concentration of 0.01% to 10% by weight.

11. The aqueous ophthalmic formulation of claim 1, further comprising a preservative which comprises butylparaben, benzalkonium chloride, benzalkonium bromide, chlorhexidine, sorbate, chlorobutanol, or any combination thereof.

12. The aqueous ophthalmic formulation of claim 10, wherein the preservative in the formulation is at a concentration of 0.01% to 5% by weight.

13. The aqueous ophthalmic formulation of claim 1, wherein the pH adjuster comprises boric acid, sodium borate, phosphate buffer, tromethamine, tromethamine hydrochloric acid buffer, sodium hydroxide, hydrochloric acid, citric acid, sodium citrate, or any combination thereof.

14. The aqueous ophthalmic formulation of claim 1, wherein the pH adjuster in the formulation is at a concentration of 0.01% to 5% by weight.

15. The aqueous ophthalmic formulation of claim 1, wherein the viscosity adjuster in the formulation has a concentration of 0.01% to 5% by weight.

16. The aqueous ophthalmic formulation of claim 1, wherein the viscosity adjuster comprises carboxyl methyl cellulose, sodium cellulose, hydroxypropyl cellulose, hydroxyethyl cellulose, or any combination thereof.

17. The aqueous ophthalmic formulation of claim 1, wherein the average particle size of the micelles ranges from 10 nm to 20 nm.

18. A micelle comprising water, cyclosporine A, and a solubilizer, wherein the micelle has a particle size no greater than 20 nm.

19. The micelle of claim 18, wherein the solubilizer comprises Polyoxyl 20 Cetostearyl Ether, Polyoxyl 15 Hydroxystearate, Soluplus, Polyoxyethylene hydrogenated castor oil, Polyoxyethylene castor oil, Vitamin E Polyethylene Glycol Succinate, or any combination thereof; and the cyclosporine is cyclosporin A.

20. A method of treating or alleviating symptoms of dry eye disease or condition in a subject in need thereof, comprising topically administering to the eye of the subject a therapeutically effective amount of an aqueous ophthalmic formulation of claim 1.
